(19) United States
(12) Patent Application Publication
(10) Pub. No.: US 2025/0152052 A1
(43) Pub. Date: May 15, 2025

(54) SYSTEMS, DEVICES, AND METHODS FOR AN ANALYTE SENSOR

(71) Applicant: ABBOTT DIABETES CARE INC., Alameda, CA (US)

(72) Inventors: William MATIEVICH JR., Alameda, CA (US); Namvar KIAIE, Danville, CA (US); Timothy C. DUNN, San Francisco, CA (US)

(73) Assignee: ABBOTT DIABETES CARE INC., Alameda, CA (US)

(21) Appl. No.: 18/838,806

(22) PCT Filed: Feb. 21, 2023
(86) PCT No.: PCT/US2023/013528

§ 371 Date: Aug. 15, 2024

Related U.S. Application Data
(60) Provisional application No. 63/312,742, filed on Feb. 22, 2022.

(51) Int. Cl. A61B 5/145; A61B 5/00; A61B 5/01

(52) U.S. Cl. CPC A61B 5/14546; A61B 5/01; A61B 5/14503; A61B 5/7264; A61B 5/7405; A61B 5/742; A61B 5/7455; A61B 5/7475; A61B 2560/0252; A61B 2560/045; A61B 2560/0462; A61B 2562/0271

(57) ABSTRACT

A method comprising receiving a plurality of analyte data over a first time period monitored by an analyte sensor in fluid contact with bodily fluid under a skin surface, the plurality of analyte data corresponding to an analyte level, receiving a plurality of temperature data over the first time period from a temperature sensor, determining a rate of change of the plurality of temperature data over the first time period, if the determined rate of change of the plurality of temperature data is above a predetermined threshold, receiving user input to confirm exposure to radiologic procedure during the first time period, and adjusting the plurality of analyte data over the first time period based on the confirmed exposure to radiologic procedure.

### CROSS-REFERENCE TO RELATED APPLICATIONS

This application claims the benefit, under 35 U.S.C. § 119 (e), of U.S. Provisional Patent Application No. 63/312,742, filed Feb. 22, 2022, which is incorporated herein by reference in its entirety and for all purposes.

### FIELD

The subject matter described herein relates generally to systems, devices, and methods for analyte sensors. For example, systems, devices, and methods for mitigating the impact of radiologic procedures on on-body sensor puck assembly and measured analyte levels.

### BACKGROUND

The detection and/or monitoring of analyte levels, such as glucose, ketones, lactate, oxygen, hemoglobin AIC, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting a sensor, such as a dermal sensor that senses a user's analyte level in a bodily fluid located in the dermal layer of the human body, using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While present continuous glucose monitors and sensors control devices can be convenient for users, they can be made more useful by being adapted for use during a magnetic resonance imaging (MRI) or other radiological diagnostic procedures.

Magnetic resonance imaging (MRI) is an effective, non-invasive imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed radio-frequency (RF) magnetic field is then applied causing precession of the protons around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

A significant problem with MRI is that its strong magnetic fields can interfere with the operation of continuous glucose monitors (CGM). Typically, CGMs include a sensor control device positioned on a human body with a sensor in contact with the wearer's bodily fluid to measure analyte levels. Magnetic fields from MRI may cause changes in the functionality of CGMs. Therefore, wearers are generally advised to avoid exposure to MRIs.

However, it would be preferable to allow user to continue wearing the CGM device even during an MRI procedure, so long as any inaccurate CGM readings are addressed before being reported to the user. Accordingly, there is a need for systems, devices, and methods for mitigating the impact of radiologic procedures on sensor control devices, sensors, and measured analyte levels.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to a method including receiving a plurality of analyte data for a first time period monitored by an analyte sensor in fluid contact with bodily fluid under a skin surface, the plurality of analyte data corresponding to an analyte level, receiving a plurality of temperature data for the first time period from a temperature sensor, determining a rate of change of the plurality of temperature data over the first time period, if the determined rate of change of the plurality of temperature data is above a predetermined threshold, receiving user input to confirm exposure to radiologic procedure during the first time period, and adjusting the plurality of analyte data over the first time period based on the confirmed exposure to radiologic procedure.

According to embodiments, the time period can include one hour. The predetermined threshold can be 4 degrees Celsius over 15 minutes. The user input can be received via a reader device.

According to embodiments, adjusting the plurality of analyte data can include removing the plurality of analyte data for the first time period. Adjusting the plurality of analyte data can include ignoring the plurality of analyte data for the first time period.

According to embodiments, the temperature data can comprise on-skin temperature data. According to embodiments, confirming exposure to the radiologic procedure can include at least one of prompting and generating an alarm.

According to embodiments, the method can include receiving a user input indicating a first time period corresponding to anticipated exposure to a radiologic procedure, receiving a plurality of analyte data over the first time period monitored by an analyte sensor in fluid contact with bodily fluid under a skin surface, the plurality of analyte data corresponding to an analyte level, receiving a plurality of temperature data over the first time period for the skin surface from a temperature sensor, determining a rate of change of the plurality of temperature data over the first time period, confirming exposure to radiologic procedure during the first time period if the determined rate of change of the plurality of temperature data is above a predetermined threshold, and adjusting the plurality of analyte data over the first time period based on the confirmed exposure to radiologic procedure.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIGS. 5A and 5B are isometric and side views, respectively, of another example sensor control device.

FIGS. 6A and 6F are exploded isometric top and bottom views, respectively of the sensor control device of FIGS. 5A-5B.

FIG. 7 is a cross-sectional side view of an assembled sealed subassembly, according to one or more embodiments.

FIGS. 8A-8C are progressive cross-sectional side views showing assembly of the sensor applicator with the sensor control device of FIGS. 5A-5B.

FIGS. 9A and 9B are perspective and top views, respectively, of the cap post of FIG. 21C, according to one or more additional embodiments.

FIG. 10 is a cross-sectional side view of the sensor control device of FIGS. 18A-18B.

FIGS. 11A and 11B are cross-sectional side views of the sensor applicator ready to deploy the sensor control device to a target monitoring location.

FIGS. 12A-12C are progressive cross-sectional side views showing assembly and disassembly of an example embodiment of the sensor applicator with the sensor control device of FIGS. 5A-5B.

FIGS. 13A and 13B are side and isometric views, respectively, of an example sensor control device, according to one or more embodiments of the present disclosure.

FIGS. 14A and 14B are exploded, isometric top and bottom views, respectively, of the sensor control device of FIG. 2, according to one or more embodiments.

FIG. 15 is a cross-sectional side view of the sensor control device of FIGS. 31A-31B and 14A-14B, according to one or more embodiments.

FIG. 16A is an exploded isometric view of a portion of another embodiment of the sensor control device of FIGS. 13A-13B and 14A-14B.

FIG. 17A is an isometric bottom view of the mount of FIGS. 13A-13B and 32A-32B.

FIG. 17B is an isometric top view of the sensor cap of FIGS. 13A-13B and 32A-32B.

FIGS. 18A and 18B are side and cross-sectional side views, respectively, of an example sensor applicator, according to one or more embodiments.

FIG. 19 shows exemplary change in glucose readings before, during, and after exposure to magnetic resonance imagining protocols.

FIG. 20 shows summary of MRI RF-induced heating results in one or more embodiments.

FIG. 21 shows summary of MRI gradient-induced heating results in one or more embodiments.

FIG. 22 shows measured maximum artifact of exemplary embodiments at 3T for gradient echo and spin echo sequences.

FIGS. 23 and 24 shows flowcharts illustrating exemplary routines associated with determining exposure to radiologic procedure according to one or more embodiments.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for the improved assembly and use of dermal sensor insertion devices for use with in vivo analyte monitoring systems. In particular, several embodiments of the present disclosure are designed to improve the method of sensor insertion with respect to in vivo analyte monitoring systems and, in particular, to prevent the premature retraction of an insertion sharp during a sensor insertion process. Some embodiments, for example, include a dermal sensor insertion mechanism with an increased firing velocity and a delayed sharp retraction. In other embodiments, the sharp retraction mechanism can be motion-actuated such that the sharp is not retracted until the user pulls the applicator away from the skin. Consequently, these embodiments can reduce the likelihood of prematurely withdrawing an insertion sharp during a sensor insertion process; decrease the likelihood of improper sensor insertion; and decrease the likelihood of damaging a sensor during the sensor insertion process, to name a few advantages. Several embodiments of the present disclosure also provide for improved insertion sharp modules to account for the small scale of dermal sensors and the relatively shallow insertion path present in a subject's dermal layer. In addition, several embodiments of the present disclosure are designed to prevent undesirable axial and/or rotational movement of applicator components during sensor insertion. Accordingly, these embodiments can reduce the likelihood of instability of a positioned dermal sensor, irritation at the insertion site, damage to surrounding tissue, and breakage of capillary blood vessels resulting in fouling of the dermal fluid with blood, to name a few advantages. In addition, to mitigate inaccurate sensor readings which can be caused by trauma at the insertion site, several embodiments of the present disclosure can reduce the end-depth penetration of the needle relative to the sensor tip during insertion.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

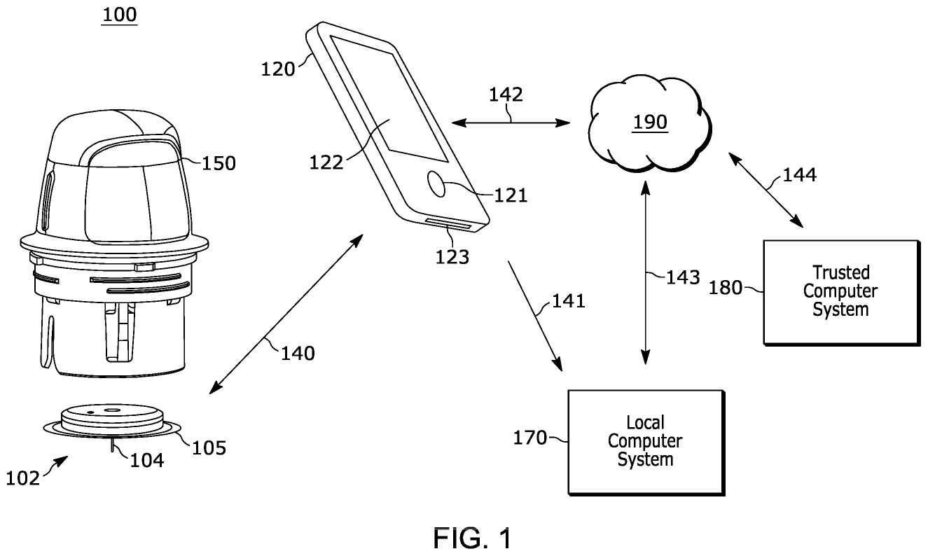

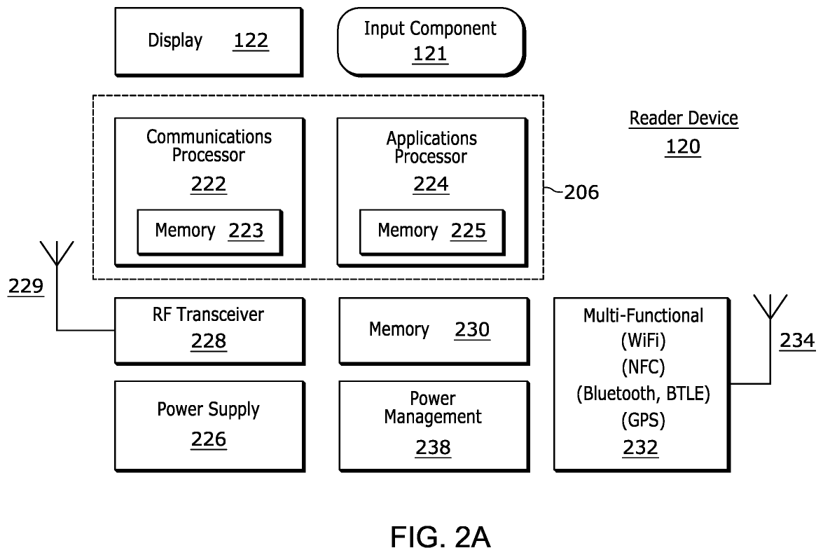

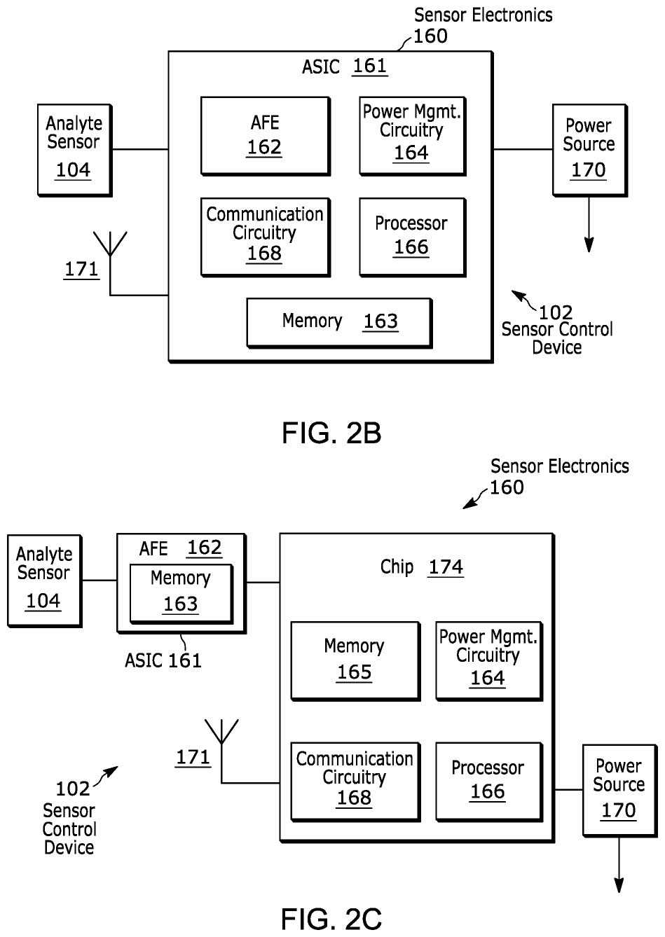

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiment of Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Device

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

According to embodiments, the sensor control device 102 can include a temperature sensor for measurement of skin near the insertion site. The temperature readings may be used to adjust the measurement data generated from the analyte sensor 104, as discussed in further detail below. Additional details of suitable devices, systems, methods, components and the operation thereof along with related features are set forth in U.S. Patent Publication No. 2019/0069823, filed Nov. 5, 2018, which is incorporated by reference in its entirety herein.

### Example Embodiment of Assembly Process for Sensor Control Device

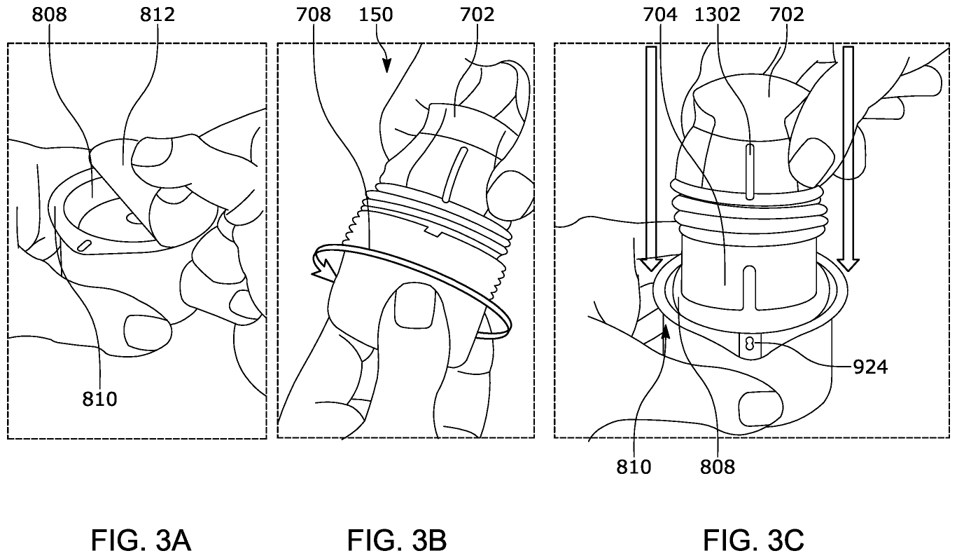

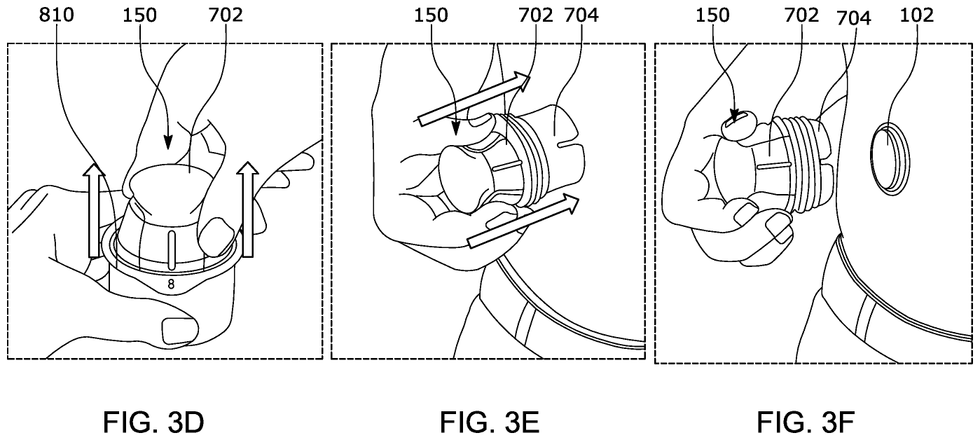

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 810, configured here as a tray (although other packages can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting an applicator device 150 into a tray 810 during an assembly. Initially, the user can insert sheath 704 into platform 808 inside tray 810 after aligning housing orienting feature 1302 (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from a tray 810 during an assembly. A user can remove applicator 150 from tray 810 by proximally advancing housing 702 with respect to tray 810 or other motions having the same end effect of uncoupling applicator 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Example Embodiment of Sensor Applicator Device

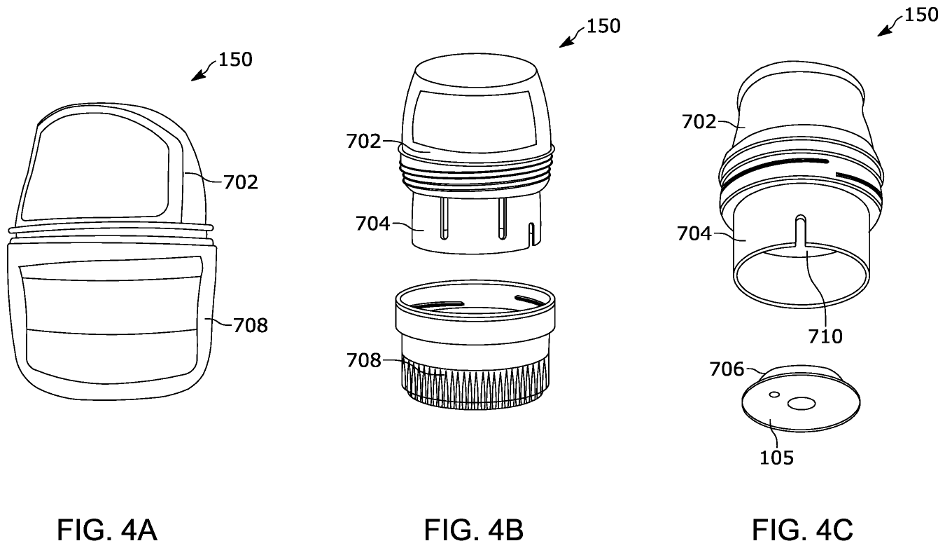

FIG. 4A is a side view depicting an example embodiment of an applicator device 150 coupled with screw cap 708. This is an example of how applicator 150 is shipped to and received by a user, prior to assembly by the user with a sensor. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within sensor electronics carrier 710 of sheath 704, when cap 708 is in place.

### Example Embodiments of Applicators and Sensor Control Devices for One Piece Architectures

Referring briefly again to FIGS. 1 and 3A-3G, for the two-piece architecture system, the sensor tray 202 and the sensor applicator 102 are provided to the user as separate packages, thus requiring the user to open each package and finally assemble the system. In some applications, the discrete, sealed packages allow the sensor tray 202 and the sensor applicator 102 to be sterilized in separate sterilization processes unique to the contents of each package and otherwise incompatible with the contents of the other. More specifically, the sensor tray 202, which includes the plug assembly 207, including the sensor 110 and the sharp 220, may be sterilized using radiation sterilization, such as electron beam (or “e-beam”) irradiation. Radiation sterilization, however, can damage the electrical components arranged within the electronics housing of the sensor control device 102. Consequently, if the sensor applicator 102, which contains the electronics housing of the sensor control device 102, needs to be sterilized, it may be sterilized via another method, such as gaseous chemical sterilization using, for example, ethylene oxide. Gaseous chemical sterilization, however, can damage the enzymes or other chemistry and biologies included on the sensor 110. Because of this sterilization incompatibility, the sensor tray 202 and the sensor applicator 102 are commonly sterilized in separate sterilization processes and subsequently packaged separately, which requires the user to finally assemble the components for use.

According to embodiments of the present disclosure, the sensor control device 102 may be modified to provide a one-piece architecture that may be subjected to sterilization techniques specifically designed for a one-piece architecture sensor control device. A one-piece architecture allows the sensor applicator 150 and the sensor control device 102 to be shipped to the user in a single, sealed package that does not require any final user assembly steps. Rather, the user need only open one package and subsequently deliver the sensor control device 102 to the target monitoring location. The one-piece system architecture described herein may prove advantageous in eliminating component parts, various fabrication process steps, and user assembly steps. As a result, packaging and waste are reduced, and the potential for user error or contamination to the system is mitigated.

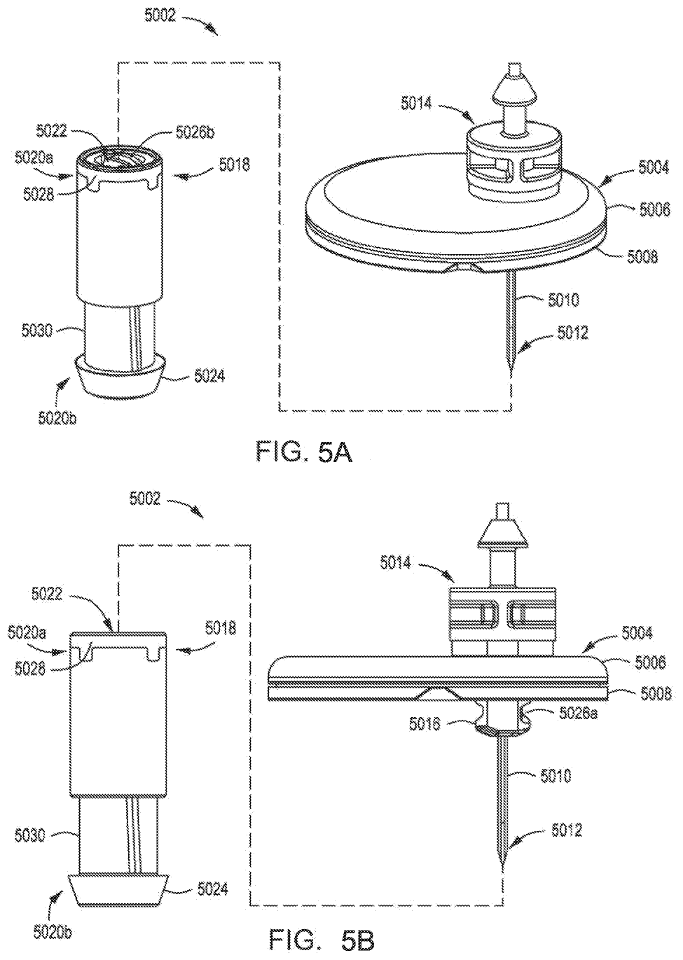

FIGS. 5A and 5B are isometric and side views, respectively, of another example sensor control device 5002, according to one or more embodiments of the present disclosure. The sensor control device 5002 may be similar in some respects to the sensor control device 102 of FIG. 1 and therefore may be best understood with reference thereto. Moreover, the sensor control device 5002 may replace the sensor control device 102 of FIG. 1 and, therefore, may be used in conjunction with the sensor applicator 102 of FIG. 1, which may deliver the sensor control device 5002 to a target monitoring location on a user's skin.

Unlike the sensor control device 102 of FIG. 1, however, the sensor control device 5002 may comprise a one-piece system architecture not requiring a user to open multiple packages and finally assemble the sensor control device 5002 prior to application. Rather, upon receipt by the user, the sensor control device 5002 may already be fully assembled and properly positioned within the sensor applicator 150 (FIG. 1). To use the sensor control device 5002, the user need only open one barrier (e.g., the applicator cap 708 of FIG. 3B) before promptly delivering the sensor control device 5002 to the target monitoring location for use.

As illustrated, the sensor control device 5002 includes an electronics housing 5004 that is generally disc-shaped and may have a circular cross-section. In other embodiments, however, the electronics housing 2004 may exhibit other cross-sectional shapes, such as ovoid or polygonal, without departing from the scope of the disclosure. The electronics housing 5004 may be configured to house or otherwise contain various electrical components used to operate the sensor control device 5002. In at least one embodiment, an adhesive patch (not shown) may be arranged at the bottom of the electronics housing 5004. The adhesive patch may be similar to the adhesive patch 105 of FIG. 1, and may thus help adhere the sensor control device 5002 to the user's skin for use.

As illustrated, the sensor control device 5002 includes an electronics housing 5004 that includes a shell 5006 and a mount 5008 that is matable with the shell 5006. The shell 5006 may be secured to the mount 5008 via a variety of ways, such as a snap fit engagement, an interference fit, sonic welding, one or more mechanical fasteners (e.g., screws), a gasket, an adhesive, or any combination thereof. In some cases, the shell 5006 may be secured to the mount 5008 such that a sealed interface is generated therebetween.

The sensor control device 5002 may further include a sensor 5010 (partially visible) and a sharp 5012 (partially visible), used to help deliver the sensor 5010 transcutaneously under a user's skin during application of the sensor control device 5002. As illustrated, corresponding portions of the sensor 5010 and the sharp 5012 extend distally from the bottom of the electronics housing 5004 (e.g., the mount 5008). The sharp 5012 may include a sharp hub 5014 configured to secure and carry the sharp 5012. As best seen in FIG. 5B, the sharp hub 5014 may include or otherwise define a mating member 5016. To couple the sharp 5012 to the sensor control device 5002, the sharp 5012 may be advanced axially through the electronics housing 5004 until the sharp hub 5014 engages an upper surface of the shell 5006 and the mating member 5016 extends distally from the bottom of the mount 5008. As the sharp 5012 penetrates the electronics housing 5004, the exposed portion of the sensor 5010 may be received within a hollow or recessed (arcuate) portion of the sharp 5012. The remaining portion of the sensor 5010 is arranged within the interior of the electronics housing 5004.

The sensor control device 5002 may further include a sensor cap 5018, shown exploded or detached from the electronics housing 5004 in FIGS. 5A-5B. The sensor cap 5016 may be removably coupled to the sensor control device 5002 (e.g., the electronics housing 5004) at or near the bottom of the mount 5008. The sensor cap 5018 may help provide a sealed barrier that surrounds and protects the exposed portions of the sensor 5010 and the sharp 5012 from gaseous chemical sterilization. As illustrated, the sensor cap 5018 may comprise a generally cylindrical body having a first end 5020a and a second end 5020b opposite the first end 5020a. The first end 5020a may be open to provide access into an inner chamber 5022 defined within the body. In contrast, the second end 5020b may be closed and may provide or otherwise define an engagement feature 5024. As described herein, the engagement feature 5024 may help mate the sensor cap 5018 to the cap (e.g., the applicator cap 708 of FIG. 3B) of a sensor applicator (e.g., the sensor applicator 150 of FIGS. 1 and 3A-3G), and may help remove the sensor cap 5018 from the sensor control device 5002 upon removing the cap from the sensor applicator.

The sensor cap 5018 may be removably coupled to the electronics housing 5004 at or near the bottom of the mount 5008. More specifically, the sensor cap 5018 may be removably coupled to the mating member 5016, which extends distally from the bottom of the mount 5008. In at least one embodiment, for example, the mating member 5016 may define a set of external threads 5026a (FIG. 5B) matable with a set of internal threads 5026b (FIG. 5A) defined by the sensor cap 5018. In some embodiments, the external and internal threads 5026a, b may comprise a flat thread design (e.g., lack of helical curvature), which may prove advantageous in molding the parts. Alternatively, the external and internal threads 5026a,b may comprise a helical threaded engagement. Accordingly, the sensor cap 5018 may be threadably coupled to the sensor control device 5002 at the mating member 5016 of the sharp hub 5014. In other embodiments, the sensor cap 5018 may be removably coupled to the mating member 5016 via other types of engagements including, but not limited to, an interference or friction fit, or a frangible member or substance that may be broken with minimal separation force (e.g., axial or rotational force).

In some embodiments, the sensor cap 5018 may comprise a monolithic (singular) structure extending between the first and second ends 5020a, b. In other embodiments, however, the sensor cap 5018 may comprise two or more component parts. In the illustrated embodiment, for example, the sensor cap 5018 may include a seal ring 5028 positioned at the first end 5020a and a desiccant cap 5030 arranged at the second end 5020b. The seal ring 5028 may be configured to help seal the inner chamber 5022, as described in more detail below. In at least one embodiment, the seal ring 5028 may comprise an elastomeric O-ring. The desiccant cap 5030 may house or comprise a desiccant to help maintain preferred humidity levels within the inner chamber 5022. The desiccant cap 5030 may also define or otherwise provide the engagement feature 5024 of the sensor cap 5018.

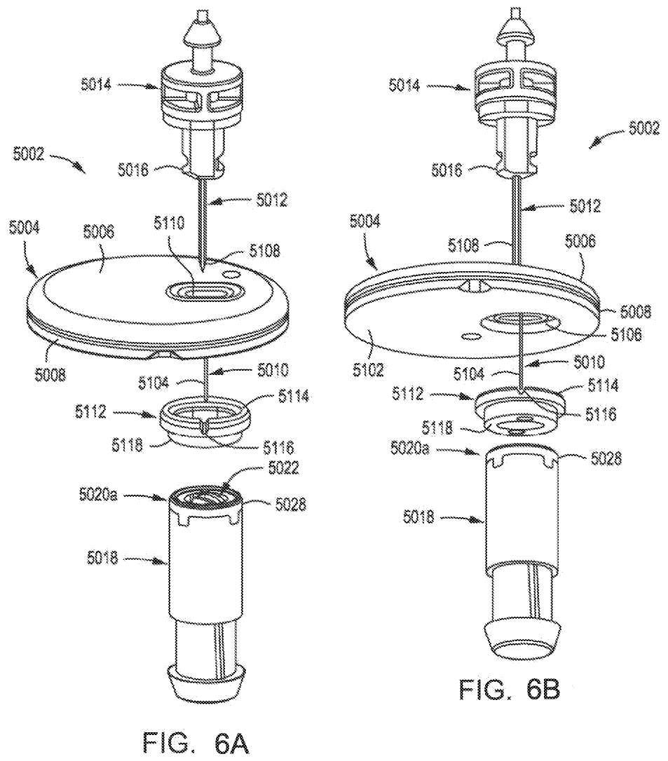

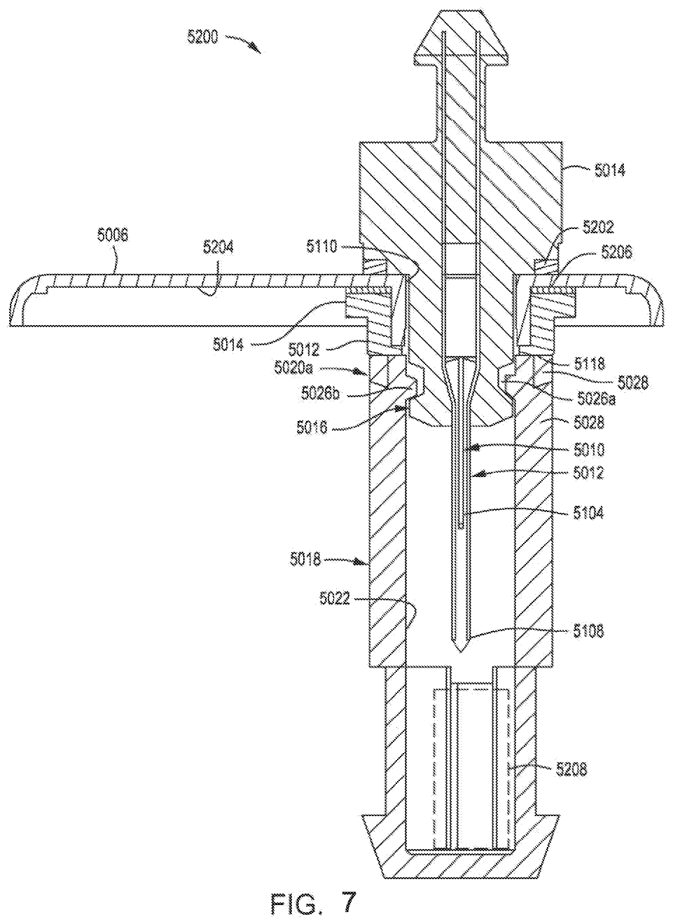

FIGS. 6A and 6B are exploded isometric top and bottom views, respectively, of the sensor control device 5002, according to one or more embodiments. The shell 5006 and the mount 5008 operate as opposing clamshell halves that enclose or otherwise substantially encapsulate various electronic components of the sensor control device 5002. More specifically, electronic components may include, but are not limited to, a printed circuit board (PCB), one or more resistors, transistors, capacitors, inductors, diodes, and switches. A data processing unit and a battery may be mounted to or otherwise interact with the PCB. The data processing unit may comprise, for example, an application specific integrated circuit (ASIC) configured to implement one or more functions or routines associated with operation of the sensor control device 5002. More specifically, the data processing unit may be configured to perform data processing functions, where such functions may include, but are not limited to, filtering and encoding of data signals, each of which corresponds to a sampled analyte level of the user. The data processing unit may also include or otherwise communicate with an antenna for communicating with the reader device 120 (FIG. 1). The battery may provide power to the sensor control device 5002 and, more particularly, to the electronic components of the PCB. While not shown, the sensor control device 5002 may also include an adhesive patch that may be applied to the bottom 5102 (FIG. 6B) of the mount 5008, and may help adhere the sensor control device 5002 to the user's skin for use.

The sensor control device 5002 may provide or otherwise include a sealed subassembly that includes, among other component parts, the shell 5006, the sensor 5010, the sharp 5012, and the sensor cap 5018. The sealed subassembly of the sensor control device 5002 may help isolate the sensor 5010 and the sharp 5012 within the inner chamber 5022 (FIG. 6A) of the sensor cap 5018 during a gaseous chemical sterilization process, which might otherwise adversely affect the chemistry provided on the sensor 5010.

The sensor 5010 may include a tail 5104 that extends out an aperture 5106 (FIG. 6B) defined in the mount 5008 to be transcutaneously received beneath a user's skin. The tail 5104 may have an enzyme or other chemistry included thereon to help facilitate analyte monitoring. The sharp 5012 may include a sharp tip 5108 extendable through an aperture 5110 (FIG. 51A) defined by the shell 5006, and the aperture 5110 may be coaxially aligned with the aperture 5106 of the mount 5008. As the sharp tip 5108 penetrates the electronics housing 5004, the tail 5104 of the sensor 5010 may be received within a hollow or recessed portion of the sharp tip 5108. The sharp tip 5108 may be configured to penetrate the skin while carrying the tail 5104 to put the active chemistry of the tail 5104 into contact with bodily fluids.

The sharp tip 5108 may be advanced through the electronics housing 5004 until the sharp hub 5014 engages an upper surface of the shell 5006 and the mating member 5016 extends out the aperture 5106 in the bottom 5102 of the mount 5008. In some embodiments, a seal member (not shown), such as an O-ring or seal ring, may interpose the sharp hub 5014 and the upper surface of the shell 5006 to help seal the interface between the two components. In some embodiments, the seal member may comprise a separate component part, but may alternatively form an integral part of the shell 5006, such as being a co-molded or overmolded component part.

The sealed subassembly may further include a collar 5112 that is positioned within the electronics housing 5004 and extends at least partially into the aperture 5106. The collar 5112 may be a generally annular structure that defines or otherwise provides an annular ridge 5114 on its top surface. In some embodiments, as illustrated, a groove 5116 may be defined in the annular ridge 5114 and may be configured to accommodate or otherwise receive a portion of the sensor 5010 extending laterally within the electronics housing 5004.

In assembling the sealed subassembly, a bottom 5118 of the collar 5112 may be exposed at the aperture 5106 and may sealingly engage the first end 5020a of the sensor cap 5018 and, more particularly, the seal ring 5028. In contrast, the annular ridge 5114 at the top of the collar 5112 may sealingly engage an inner surface (not shown) of the shell 5006. In at least one embodiment, a seal member (not shown) may interpose the annular ridge 5114 and the inner surface of the shell 5006 to form a sealed interface. In such embodiments, the seal member may also extend (flow) into the groove 5116 defined in the annular ridge 5114 and thereby seal about the sensor 5010 extending laterally within the electronics housing 5004. The seal member may comprise, for example, an adhesive, a gasket, or an ultrasonic weld, and may help isolate the enzymes and other chemistry included on the tail 5104.

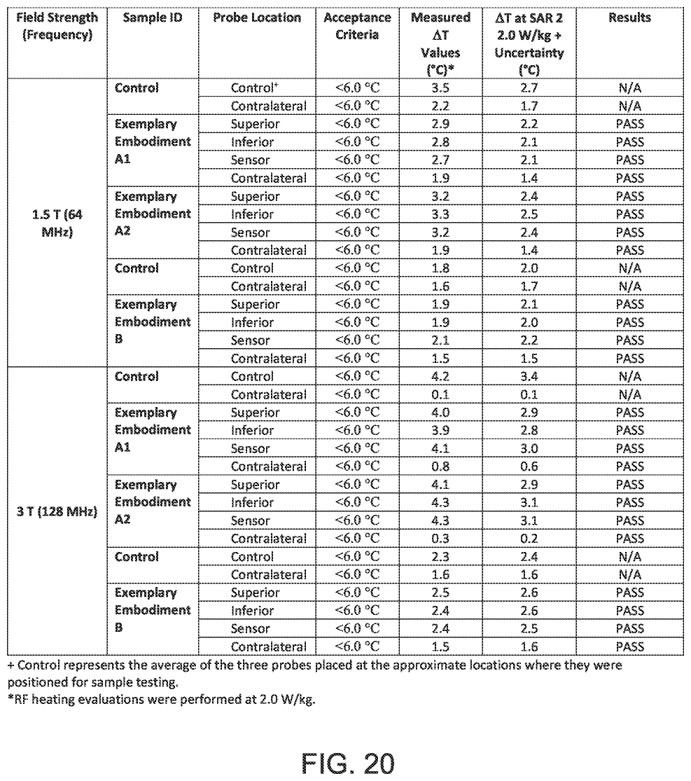

FIG. 20 is a cross-sectional side view of an assembled sealed subassembly 5200, according to one or more embodiments. The sealed subassembly 5200 may form part of the sensor control device 5002 of FIGS. 5A-5B and 6A-6B and may include portions of the shell 5006, the sensor 5010, the sharp 5012, the sensor cap 5018, and the collar 5112. The sealed subassembly 5200 may be assembled in a variety of ways. In one assembly process, the sharp 5012 may be coupled to the sensor control device 5002 by extending the sharp tip 5108 through the aperture 5110 defined in the top of the shell 5006 and advancing the sharp 5012 through the shell 5006 until the sharp hub 5014 engages the top of the shell 5006 and the mating member 196 extends distally from the shell 5006. In some embodiments, as mentioned above, a seal member 5202 (e.g., an O-ring or seal ring) may interpose the sharp hub 5014 and the upper surface of the shell 5006 to help seal the interface between the two components.

The collar 5112 may then be received over (about) the mating member 5016 and advanced toward an inner surface 5204 of the shell 5006 to enable the annular ridge 5114 to engage the inner surface 5204. A seal member 5206 may interpose the annular ridge 5114 and the inner surface 5204 and thereby form a sealed interface. The seal member 5206 may also extend (flow) into the groove 5116 (FIGS. 6A-6B) defined in the annular ridge 5114 and thereby seal about the sensor 5010 extending laterally within the electronics housing 5004 (FIGS. 6A-6B). In other embodiments, however, the collar 5112 may first be sealed to the inner surface 5204 of the shell 5006, following which the sharp 5012 and the sharp hub 5014 may be extended through the aperture 5110, as described above.

The sensor cap 5018 may be removably coupled to the sensor control device 5002 by threadably mating the internal threads 5026b of the sensor cap 5018 with the external threads 5026a of the mating member 5016. Tightening (rotating) the mated engagement between the sensor cap 5018 and the mating member 5016 may urge the first end 5020a of the sensor cap 5018 into sealed engagement with the bottom 5118 of the collar 5112. Moreover, tightening the mated engagement between the sensor cap 5018 and the mating member 5016 may also enhance the sealed interface between the sharp hub 5014 and the top of the shell 5006, and between the annular ridge 5114 and the inner surface 5204 of the shell 5006.

The inner chamber 5022 may be sized and otherwise configured to receive the tail 5104 and the sharp tip 5108. Moreover, the inner chamber 5022 may be sealed to isolate the tail 5104 and the sharp tip 5108 from substances that might adversely interact with the chemistry of the tail 5104. In some embodiments, a desiccant 5208 (shown in dashed lines) may be present within the inner chamber 5022 to maintain proper humidity levels.

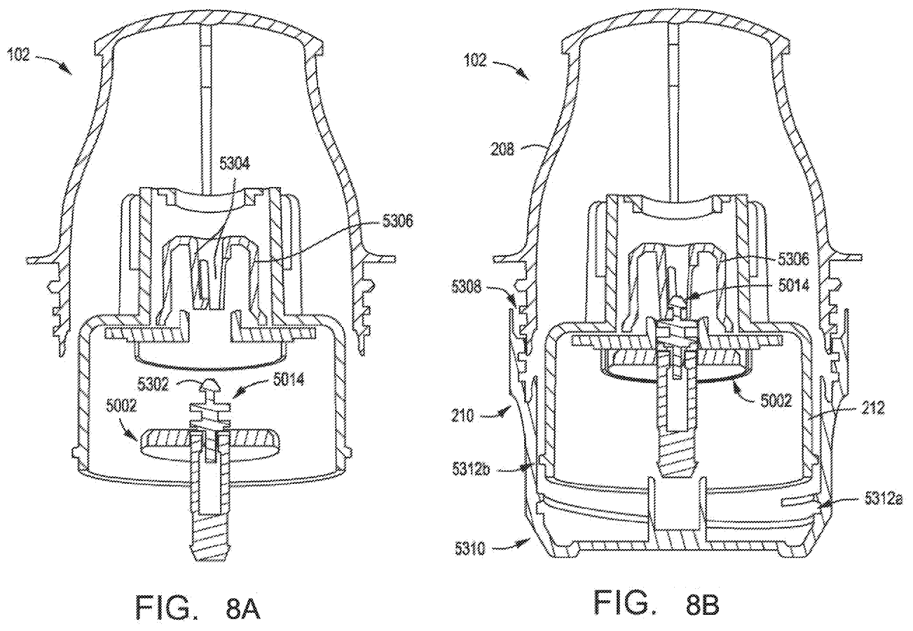

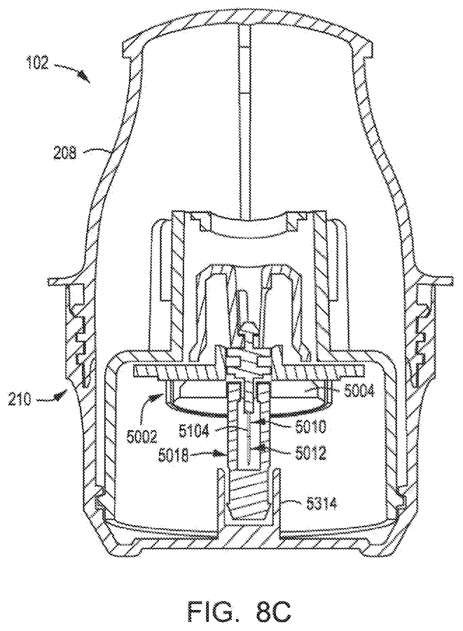

FIGS. 8A-8C are progressive cross-sectional side views showing assembly of the sensor applicator 102 with the sensor control device 5002, according to one or more embodiments. Once the sensor control device 5002 is fully assembled, it may then be loaded into the sensor applicator 102. With reference to FIG. 8A, the sharp hub 5014 may include or otherwise define a hub snap pawl 5302 configured to help couple the sensor control device 5002 to the sensor applicator 102. More specifically, the sensor control device 5002 may be advanced into the interior of the sensor applicator 102 and the hub snap pawl 5302 may be received by corresponding arms 5304 of a sharp carrier 5306 positioned within the sensor applicator 102.

In FIG. 8B, the sensor control device 5002 is shown received by the sharp carrier 5306 and, therefore, secured within the sensor applicator 102. Once the sensor control device 5002 is loaded into the sensor applicator 102, the applicator cap 210 may be coupled to the sensor applicator 102. In some embodiments, the applicator cap 210 and the housing 208 may have opposing, matable sets of threads 5308 that enable the applicator cap 210 to be screwed onto the housing 208 in a clockwise (or counter-clockwise) direction and thereby secure the applicator cap 210 to the sensor applicator 102.

As illustrated, the sheath 212 is also positioned within the sensor applicator 102, and the sensor applicator 102 may include a sheath locking mechanism 5310 configured to ensure that the sheath 212 does not prematurely collapse during a shock event. In the illustrated embodiment, the sheath locking mechanism 5310 may comprise a threaded engagement between the applicator cap 210 and the sheath 212. More specifically, one or more internal threads 53 12a may be defined or otherwise provided on the inner surface of the applicator cap 210, and one or more external threads 53 12b may be defined or otherwise provided on the sheath 212. The internal and external threads 53 12a,b may be configured to threadably mate as the applicator cap 210 is threaded to the sensor applicator 102 at the threads 5308. The internal and external threads 53 12a,b may have the same thread pitch as the threads 5308 that enable the applicator cap 210 to be screwed onto the housing 208.

In FIG. 8C, the applicator cap 210 is shown fully threaded (coupled) to the housing 208. As illustrated, the applicator cap 210 may further provide and otherwise define a cap post 5314 centrally located within the interior of the applicator cap 210 and extending proximally from the bottom thereof. The cap post 5314 may be configured to receive at least a portion of the sensor cap 5018 as the applicator cap 210 is screwed onto the housing 208.

With the sensor control device 5002 loaded within the sensor applicator 102 and the applicator cap 210 properly secured, the sensor control device 5002 may then be subjected to a gaseous chemical sterilization configured to sterilize the electronics housing 5004 and any other exposed portions of the sensor control device 5002. Since the distal portions of the sensor 5010 and the sharp 5012 are sealed within the sensor cap 5018, the chemicals used during the gaseous chemical sterilization process are unable to interact with the enzymes, chemistry, and biologies provided on the tail 5104, and other sensor components, such as membrane coatings that regulate analyte influx.

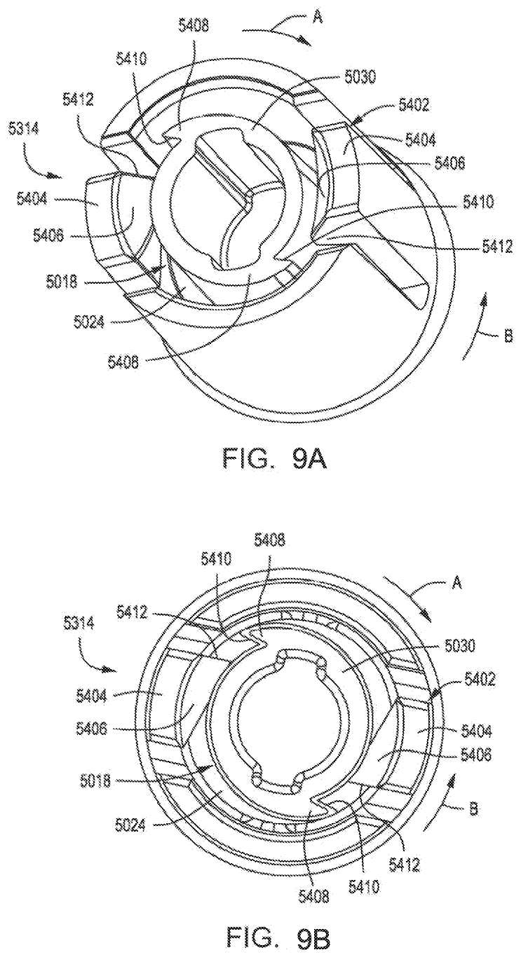

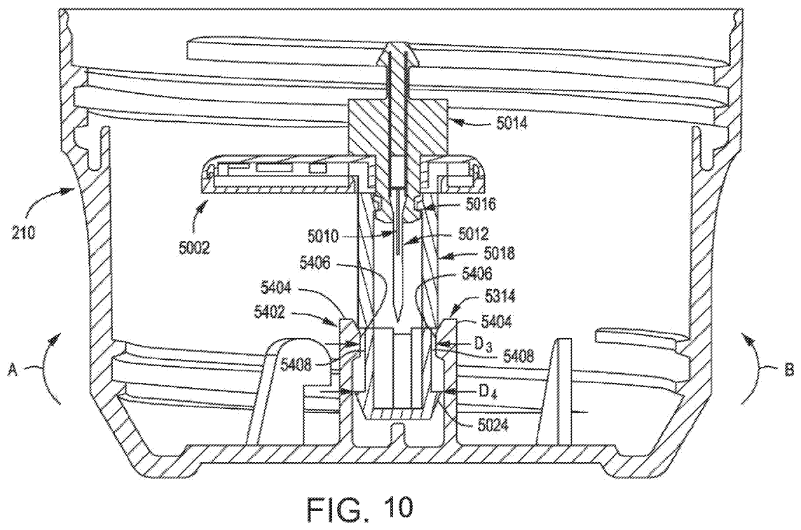

FIGS. 9A and 9B are perspective and top views, respectively, of the cap post 5314, according to one or more additional embodiments. In the illustrated depiction, a portion of the sensor cap 5018 is received within the cap post 5314 and, more specifically, the desiccant cap 5030 of the sensor cap 5018 is arranged within cap post 5314.

As illustrated, the cap post 5314 may define a receiver feature 5402 configured to receive the engagement feature 5024 of the sensor cap 5018 upon coupling (e.g., threading) the applicator cap 210 (FIG. 8C) to the sensor applicator 102 (FIGS. 8A-8C). Upon removing the applicator cap 210 from the sensor applicator 102, however, the receiver feature 5402 may prevent the engagement feature 914 from reversing direction and thus prevent the sensor cap 5018 from separating from the cap post 5314. Instead, removing the applicator cap 210 from the sensor applicator 102 will simultaneously detach the sensor cap 5018 from the sensor control device 5002 (FIGS. 5A-5B and 8A-8C), and thereby expose the distal portions of the sensor 5010 (FIGS. 8A-8C) and the sharp 5012 (FIGS. 8A-8C).

Many design variations of the receiver feature 5402 may be employed, without departing from the scope of the disclosure. In the illustrated embodiment, the receiver feature 5402 includes one or more compliant members 5404 (two shown) that are expandable or flexible to receive the engagement feature 5024 (FIGS. 5A-5B). The engagement feature 5024 may comprise, for example, an enlarged head and the compliant member(s) 5404 may comprise a collet-type device that includes a plurality of compliant fingers configured to flex radially outward to receive the enlarged head.

The compliant member(s) 5404 may further provide or otherwise define corresponding ramped surfaces 5406 configured to interact with one or more opposing camming surfaces 5408 provided on the outer wall of the engagement feature 5024. The configuration and alignment of the ramped surface(s) 5406 and the opposing camming surface(s) 5408 is such that the applicator cap 210 is able to rotate relative to the sensor cap 5018 in a first direction A (e.g., clockwise), but the cap post 5314 binds against the sensor cap 5018 when the applicator cap 210 is rotated in a second direction B (e.g., counter clockwise). More particularly, as the applicator cap 210 (and thus the cap post 5314) rotates in the first direction A, the camming surfaces 5408 engage the ramped surfaces 5406, which urge the compliant members 5404 to flex or otherwise deflect radially outward and results in a ratcheting effect. Rotating the applicator cap 210 (and thus the cap post 5314) in the second direction B, however, will drive angled surfaces 5410 of the camming surfaces 5408 into opposing angled surfaces 5412 of the ramped surfaces 5406, which results in the sensor cap 5018 binding against the compliant member(s) 5404.

FIG. 10 is a cross-sectional side view of the sensor control device 5002 positioned within the applicator cap 210, according to one or more embodiments. As illustrated, the opening to the receiver feature 5402 exhibits a first diameter D3, while the engagement feature 5024 of the sensor cap 5018 exhibits a second diameter D4 that is larger than the first diameter D3 and greater than the outer diameter of the remaining portions of the sensor cap 5018. As the sensor cap 5018 is extended into the cap post 5314, the compliant member(s) 5404 of the receiver feature 5402 may flex (expand) radially outward to receive the engagement feature 5024. In some embodiments, as illustrated, the engagement feature 5024 may provide or otherwise define an angled or frustoconical outer surface that helps bias the compliant member(s) 5404 radially outward. Once the engagement feature 5024 bypasses the receiver feature 5402, the compliant member(s) 5404 are able to flex back to (or towards) their natural state and thus lock the sensor cap 5018 within the cap post 5314.

As the applicator cap 210 is threaded to (screwed onto) the housing 208 (FIGS. 8A-8C) in the first direction A, the cap post 5314 correspondingly rotates in the same direction and the sensor cap 5018 is progressively introduced into the cap post 5314. As the cap post 5314 rotates, the ramped surfaces 5406 of the compliant members 5404 ratchet against the opposing camming surfaces 5408 of the sensor cap 5018. This continues until the applicator cap 210 is fully threaded onto (screwed onto) the housing 208. In some embodiments, the ratcheting action may occur over two full revolutions of the applicator cap 210 before the applicator cap 210 reaches its final position.

To remove the applicator cap 210, the applicator cap 210 is rotated in the second direction B, which correspondingly rotates the cap post 5314 in the same direction and causes the camming surfaces 5408 (i.e., the angled surfaces 5410 of FIGS. 9A-9B) to bind against the ramped surfaces 5406 (i.e., the angled surfaces 5412 of FIGS. 9A-9B). Consequently, continued rotation of the applicator cap 210 in the second direction B causes the sensor cap 5018 to correspondingly rotate in the same direction and thereby unthread from the mating member 5016 to allow the sensor cap 5018 to detach from the sensor control device 5002. Detaching the sensor cap 5018 from the sensor control device 5002 exposes the distal portions of the sensor 5010 and the sharp 5012, and thus places the sensor control device 5002 in position for firing (use).

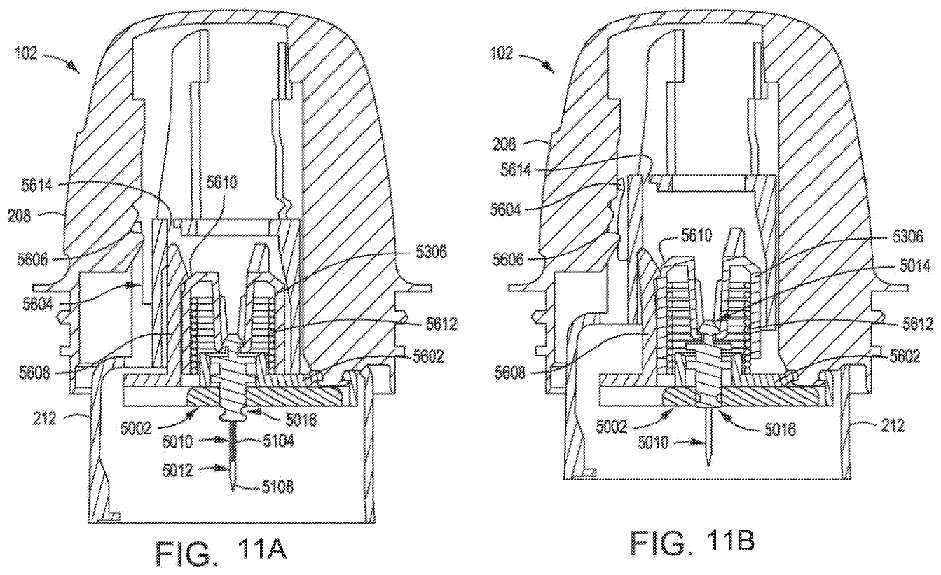

FIGS. 11A and 11B are cross-sectional side views of the sensor applicator 102 ready to deploy the sensor control device 5002 to a target monitoring location, according to one or more embodiments. More specifically, FIG. 11A depicts the sensor applicator 102 ready to deploy (fire) the sensor control device 5002, and FIG. 11B depicts the sensor applicator 102 in the process of deploying (firing) the sensor control device 5002. As illustrated, the applicator cap 210 (FIGS. 8A-8C and 55) has been removed, which correspondingly detaches (removes) the sensor cap 5018 (FIGS. 8A-8C and 55 and thereby exposes the tail 5104 of the sensor 5010 and the sharp tip 5108 of the sharp 5012, as described above. In conjunction with the sheath 212 and the sharp carrier 5306, the sensor applicator 102 also includes a sensor carrier 5602 (alternately referred to as a “puck” carrier) that helps position and secure the sensor control device 5002 within the sensor applicator 102.

Referring first to FIG. 11A, as illustrated, the sheath 212 includes one or more sheath arms 5604 (one shown) configured to interact with a corresponding one or more detents 5606 (one shown) defined within the interior of the housing 208. The detent(s) 5606 are alternately referred to as “firing” detent(s). When the sensor control device 5002 is initially installed in the sensor applicator 102, the sheath arms 5604 may be received within the detents 5606, which places the sensor applicator 102 in firing position. In the firing position, the mating member 5016 extends distally beyond the bottom of the sensor control device 5002. As discussed below, the process of firing the sensor applicator 102 causes the mating member 5016 to retract so that it does not contact the user's skin.

The sensor carrier 5602 may also include one or more carrier arms 5608 (one shown) configured to interact with a corresponding one or more grooves 5610 (one shown) defined on the sharp carrier 5306. A spring 5612 may be arranged within a cavity defined by the sharp carrier 5306 and may passively bias the sharp carrier 5306 upward within the housing 208. When the carrier arm(s) 5608 are properly received within the groove(s) 5610, however, the sharp carrier 5306 is maintained in position and prevented from moving upward. The carrier arm(s) 5608 interpose the sheath 212 and the sharp carrier 5306, and a radial shoulder 5614 defined on the sheath 212 may be sized to maintain the carrier arm(s) 5608 engaged within the groove(s) 5610 and thereby maintain the sharp carrier 5306 in position.

In FIG. 11B, the sensor applicator 102 is in the process of firing. As discussed herein with reference to FIGS. 3F-3G, this may be accomplished by advancing the sensor applicator 102 toward a target monitoring location until the sheath 212 engages the skin of the user. Continued pressure on the sensor applicator 102 against the skin may cause the sheath arm(s) 5604 to disengage from the corresponding detent(s) 5606, which allows the sheath 212 to collapse into the housing 208. As the sheath 212 starts to collapse, the radial shoulder 5614 eventually moves out of radial engagement with the carrier arm(s) 5608, which allows the carrier arm(s) 5608 to disengage from the groove(s) 5610. The passive spring force of the spring 5612 is then free to push upward on the sharp carrier 5306 and thereby force the carrier arm(s) 5608 out of engagement with the groove(s) 5610, which allows the sharp carrier 5306 to move slightly upward within the housing 208. In some embodiments, fewer coils may be incorporated into the design of the spring 5612 to increase the spring force necessary to overcome the engagement between carrier arm(s) 5608 and the groove(s) 5610. In at least one embodiment, one or both of the carrier arm(s) 5608 and the groove(s) 5610 may be angled to help ease disengagement.

As the sharp carrier 5306 moves upward within the housing 208, the sharp hub 5014 may correspondingly move in the same direction, which may cause partial retraction of the mating member 5016 such that it becomes flush, substantially flush, or sub-flush with the bottom of the sensor control device 5002. As will be appreciated, this ensures that the mating member 5016 does not come into contact with the user's skin, which might otherwise adversely impact sensor insertion, cause excessive pain, or prevent the adhesive patch (not shown) positioned on the bottom of the sensor control device 5002 from properly adhering to the skin.

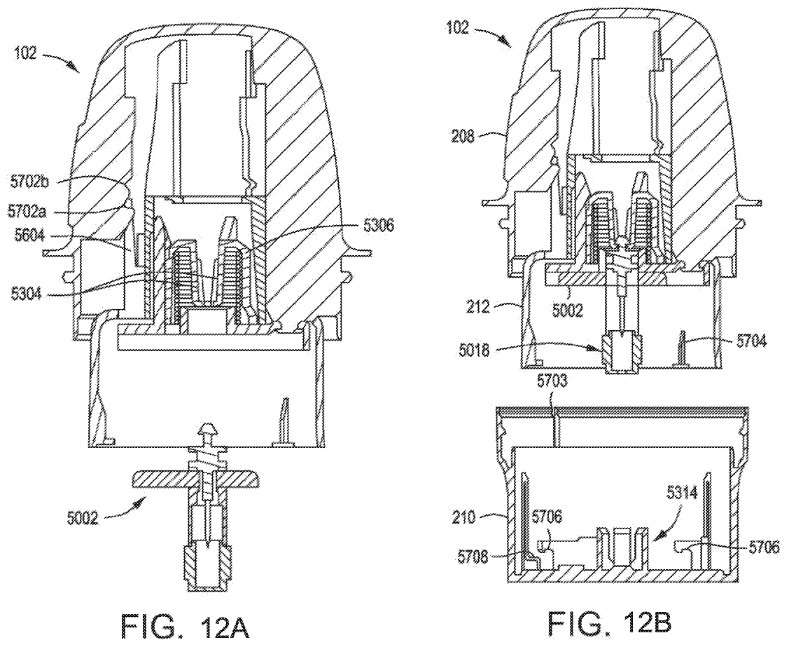

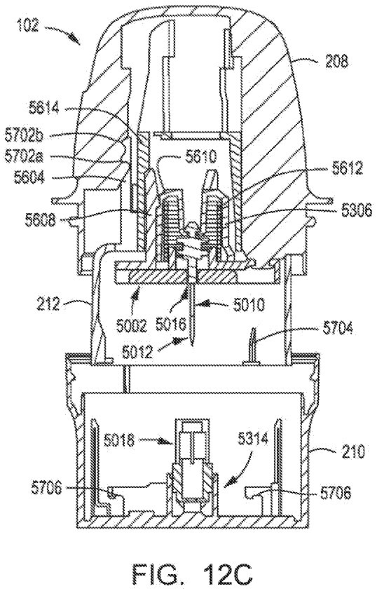

FIGS. 12A-12C are progressive cross-sectional side views showing assembly and disassembly of an alternative embodiment of the sensor applicator 102 with the sensor control device 5002, according to one or more additional embodiments. A fully assembled sensor control device 5002 may be loaded into the sensor applicator 102 by coupling the hub snap pawl 5302 into the arms 5304 of the sharp carrier 5306 positioned within the sensor applicator 102, as generally described above.

In the illustrated embodiment, the sheath arms 5604 of the sheath 212 may be configured to interact with a first detent 5702a and a second detent 5702b defined within the interior of the housing 208. The first detent 5702a may alternately be referred to a “locking” detent, and the second detent 5702b may alternately be referred to as a “firing” detent. When the sensor control device 5002 is initially installed in the sensor applicator 102, the sheath arms 5604 may be received within the first detent 5702a. As discussed below, the sheath 212 may be actuated to move the sheath arms 5604 to the second detent 5702b, which places the sensor applicator 102 in firing position.

In FIG. 12B, the applicator cap 210 is aligned with the housing 208 and advanced toward the housing 208 so that the sheath 212 is received within the applicator cap 210. Instead of rotating the applicator cap 210 relative to the housing 208, the threads of the applicator cap 210 may be snapped onto the corresponding threads of the housing 208 to couple the applicator cap 210 to the housing 208. Axial cuts or slots 5703 (one shown) defined in the applicator cap 210 may allow portions of the applicator cap 210 near its threading to flex outward to be snapped into engagement with the threading of the housing 208. As the applicator cap 210 is snapped to the housing 208, the sensor cap 5018 may correspondingly be snapped into the cap post 5314.

Similar to the embodiment of FIGS. 8A-8C, the sensor applicator 102 may include a sheath locking mechanism configured to ensure that the sheath 212 does not prematurely collapse during a shock event. In the illustrated embodiment, the sheath locking mechanism includes one or more ribs 5704 (one shown) defined near the base of the sheath 212 and configured to interact with one or more ribs 5706 (two shown) and a shoulder 5708 defined near the base of the applicator cap 210. The ribs 5704 may be configured to inter-lock between the ribs 5706 and the shoulder 5708 while attaching the applicator cap 210 to the housing 208. More specifically, once the applicator cap 210 is snapped onto the housing 208, the applicator cap 210 may be rotated (e.g., clockwise), which locates the ribs 5704 of the sheath 212 between the ribs 5706 and the shoulder 5708 of the applicator cap 210 and thereby “locks” the applicator cap 210 in place until the user reverse rotates the applicator cap 210 to remove the applicator cap 210 for use. Engagement of the ribs 5704 between the ribs 5706 and the shoulder 5708 of the applicator cap 210 may also prevent the sheath 212 from collapsing prematurely.

In FIG. 12C, the applicator cap 210 is removed from the housing 208. As with the embodiment of FIGS. 8A-8C, the applicator cap 210 can be removed by reverse rotating the applicator cap 210, which correspondingly rotates the cap post 5314 in the same direction and causes sensor cap 5018 to unthread from the mating member 5016, as generally described above. Moreover, detaching the sensor cap 5018 from the sensor control device 5002 exposes the distal portions of the sensor 5010 and the sharp 5012.

As the applicator cap 210 is unscrewed from the housing 208, the ribs 5704 defined on the sheath 212 may slidingly engage the tops of the ribs 5706 defined on the applicator cap 210. The tops of the ribs 5706 may provide corresponding ramped surfaces that result in an upward displacement of the sheath 212 as the applicator cap 210 is rotated, and moving the sheath 212 upward causes the sheath arms 5604 to flex out of engagement with the first detent 5702a to be received within the second detent 5702b. As the sheath 212 moves to the second detent 5702b, the radial shoulder 5614 moves out of radial engagement with the carrier arm(s) 5608, which allows the passive spring force of the spring 5612 to push upward on the sharp carrier 5306 and force the carrier arm(s) 5608 out of engagement with the groove(s) 5610. As the sharp carrier 5306 moves upward within the housing 208, the mating member 5016 may correspondingly retract until it becomes flush, substantially flush, or sub-flush with the bottom of the sensor control device 5002. At this point, the sensor applicator 102 in firing position. Accordingly, in this embodiment, removing the applicator cap 210 correspondingly causes the mating member 5016 to retract.

### Example Embodiments of Seal Arrangement for Analyte Monitoring Systems

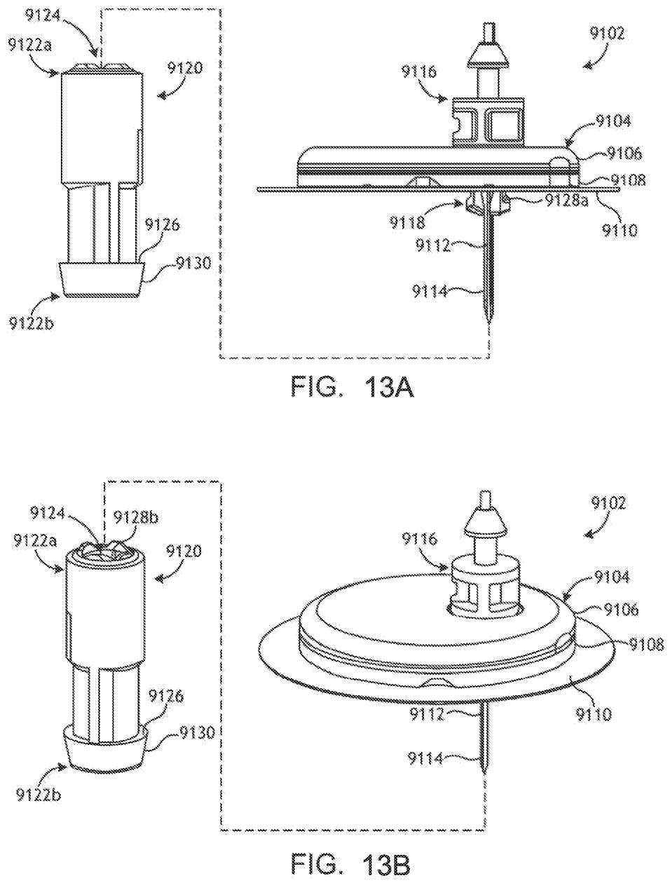

FIGS. 13A and 13B are side and isometric views, respectively, of an example sensor control device 9102, according to one or more embodiments of the present disclosure. The sensor control device 9102 may be similar in some respects to the sensor control device 102 of FIG. 1 and therefore may be best understood with reference thereto. Moreover, the sensor control device 9102 may replace the sensor control device 102 of FIG. 1 and, therefore, may be used in conjunction with the sensor applicator 102 of FIG. 1, which may deliver the sensor control device 9102 to a target monitoring location on a user's skin.

As illustrated, the sensor control device 9102 includes an electronics housing 9104, which may be generally disc-shaped and have a circular cross-section. In other embodiments, however, the electronics housing 9104 may exhibit other cross-sectional shapes, such as ovoid, oval, or polygonal, without departing from the scope of the disclosure. The electronics housing 9104 includes a shell 9106 and a mount 9108 that is matable with the shell 9106. The shell 9106 may be secured to the mount 9108 via a variety of ways, such as a snap fit engagement, an interference fit, sonic welding, laser welding, one or more mechanical fasteners (e.g., screws), a gasket, an adhesive, or any combination thereof. In some cases, the shell 9106 may be secured to the mount 9108 such that a sealed interface is generated therebetween. An adhesive patch 9110 may be positioned on and otherwise attached to the underside of the mount 9108. Similar to the adhesive patch 105 of FIG. 1, the adhesive patch 9110 may be configured to secure and maintain the sensor control device 9102 in position on the user's skin during operation.

The sensor control device 9102 may further include a sensor 9112 and a sharp 9114 used to help deliver the sensor 9112 transcutaneously under a user's skin during application of the sensor control device 9102. Corresponding portions of the sensor 9112 and the sharp 9114 extend distally from the bottom of the electronics housing 9104 (e.g., the mount 9108). A sharp hub 9116 may be overmolded onto the sharp 9114 and configured to secure and carry the sharp 9114. As best seen in FIG. 13A, the sharp hub 9116 may include or otherwise define a mating member 9118. In assembling the sharp 9114 to the sensor control device 9102, the sharp 9114 may be advanced axially through the electronics housing 9104 until the sharp hub 9116 engages an upper surface of the electronics housing 9104 or an internal component thereof and the mating member 9118 extends distally from the bottom of the mount 9108. As described herein below, in at least one embodiment, the sharp hub 9116 may sealingly engage an upper portion of a seal overmolded onto the mount 9108. As the sharp 9114 penetrates the electronics housing 9104, the exposed portion of the sensor 9112 may be received within a hollow or recessed (arcuate) portion of the sharp 9114. The remaining portion of the sensor 9112 is arranged within the interior of the electronics housing 9104.

The sensor control device 9102 may further include a sensor cap 9120, shown detached from the electronics housing 9104 in FIGS. 13A-13B. The sensor cap 9120 may help provide a sealed barrier that surrounds and protects exposed portions of the sensor 9112 and the sharp 9114. As illustrated, the sensor cap 9120 may comprise a generally cylindrical body having a first end 9122a and a second end 9122b opposite the first end 9122a. The first end 9122a may be open to provide access into an inner chamber 9124 defined within the body. In contrast, the second end 9122b may be closed and may provide or otherwise define an engagement feature 9126. As described in more detail below, the engagement feature 9126 may help mate the sensor cap 9120 to an applicator cap of a sensor applicator (e.g., the sensor applicator 102 of FIG. 1), and may help remove the sensor cap 9120 from the sensor control device 9102 upon removing the sensor cap from the sensor applicator.

The sensor cap 9120 may be removably coupled to the electronics housing 9104 at or near the bottom of the mount 9108. More specifically, the sensor cap 9120 may be removably coupled to the mating member 9118, which extends distally from the bottom of the mount 9108. In at least one embodiment, for example, the mating member 9118 may define a set of external threads 9128a (FIG. 13A) matable with a set of internal threads 9128b (FIG. 13B) defined within the inner chamber 9124 of the sensor cap 9120. In some embodiments, the external and internal threads 9128a,b may comprise a flat thread design (e.g., lack of helical curvature), but may alternatively comprise a helical threaded engagement. Accordingly, in at least one embodiment, the sensor cap 9120 may be threadably coupled to the sensor control device 9102 at the mating member 9118 of the sharp hub 9116. In other embodiments, the sensor cap 9120 may be removably coupled to the mating member 9118 via other types of engagements including, but not limited to, an interference or friction fit, or a frangible member or substance (e.g., wax, an adhesive, etc.) that may be broken with minimal separation force (e.g., axial or rotational force).

In some embodiments, the sensor cap 9120 may comprise a monolithic (singular) structure extending between the first and second ends 9122a,b. In other embodiments, however, the sensor cap 9120 may comprise two or more component parts. In the illustrated embodiment, for example, the body of the sensor cap 9120 may include a desiccant cap 9130 arranged at the second end 9122b. The desiccant cap 9130 may house or comprise a desiccant to help maintain preferred humidity levels within the inner chamber 9124. Moreover, the desiccant cap 9130 may also define or otherwise provide the engagement feature 9126 of the sensor cap 9120. In at least one embodiment, the desiccant cap 9130 may comprise an elastomeric plug inserted into the bottom end of the sensor cap 9120.

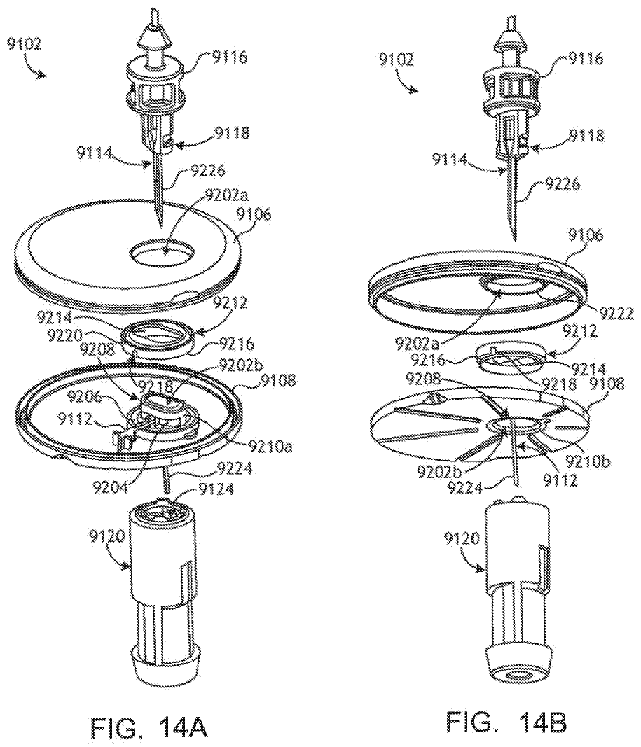

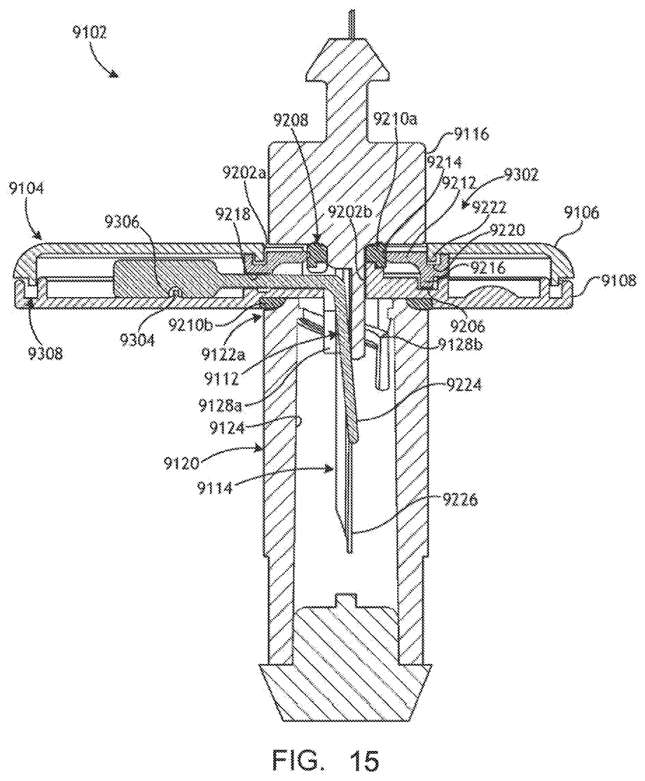

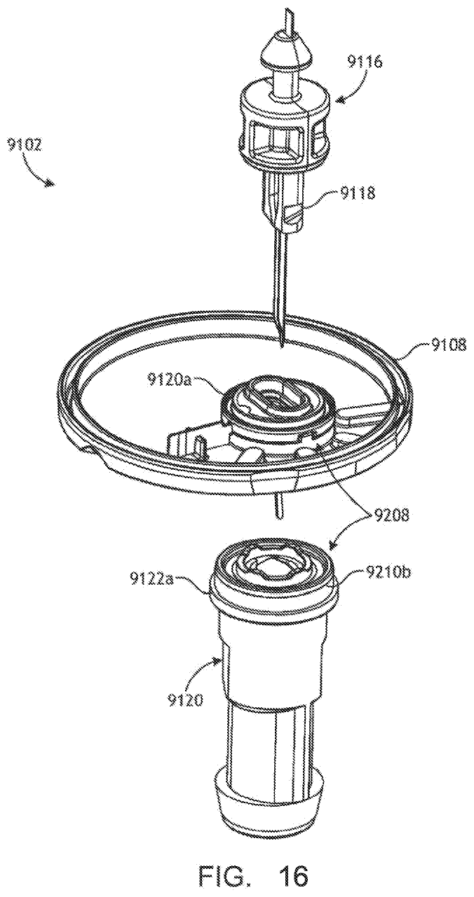

FIGS. 14A and 14B are exploded, isometric top and bottom views, respectively, of the sensor control device 9102, according to one or more embodiments. The shell 9106 and the mount 9108 operate as opposing clamshell halves that enclose or otherwise substantially encapsulate various electronic components (not shown) of the sensor control device 9102. Example electronic components that may be arranged between the shell 9106 and the mount 9108 include, but are not limited to, a battery, resistors, transistors, capacitors, inductors, diodes, and switches.

The shell 9106 may define a first aperture 9202a and the mount 9108 may define a second aperture 9202b, and the apertures 9202a, b may align when the shell 9106 is properly mounted to the mount 9108. As best seen in FIG. 14A, the mount 9108 may provide or otherwise define a pedestal 9204 that protrudes from the inner surface of the mount 9108 at the second aperture 9202b. The pedestal 9204 may define at least a portion of the second aperture 9202b. Moreover, a channel 9206 may be defined on the inner surface of the mount 9108 and may circumscribe the pedestal 9202. In the illustrated embodiment, the channel 9206 is circular in shape, but could alternatively be another shape, such as oval, ovoid, or polygonal.

The mount 9108 may comprise a molded part made of a rigid material, such as plastic or metal. In some embodiments, a seal 9208 may be overmolded onto the mount 9108 and may be made of an elastomer, rubber, a-polymer, or another pliable material suitable for facilitating a sealed interface. In embodiments where the mount 9108 is made of a plastic, the mount 9108 may be molded in a first “shot” of injection molding, and the seal 9208 may be overmolded onto the mount 9108 in a second “shot” of injection molding. Accordingly, the mount 9108 may be referred to or otherwise characterized as a “two-shot mount.”

In the illustrated embodiment, the seal 9208 may be overmolded onto the mount 9108 at the pedestal 9204 and also on the bottom of the mount 9108. More specifically, the seal 9208 may define or otherwise provide a first seal element 9210a overmolded onto the pedestal 9204, and a second seal element 9210b (FIG. 14B) interconnected to (with) the first seal element 9210a and overmolded onto the mount 9108 at the bottom of the mount 9108. In some embodiments, one or both of the seal elements 9210a,b may help form corresponding portions (sections) of the second aperture 9202b. While the seal 9208 is described herein as being overmolded onto the mount 9108, it is also contemplated herein that one or both of the seal elements 9210a,b may comprise an elastomeric component part independent of the mount 9208, such as an O-ring or a gasket.

The sensor control device 9102 may further include a collar 9212, which may be a generally annular structure that defines a central aperture 9214. The central aperture 9214 may be sized to receive the first seal element 9210a and may align with both the first and second apertures 9202a, b when the sensor control device 9102 is properly assembled. The shape of the central aperture 9214 may generally match the shape of the second aperture 9202b and the first seal element 9210a.

In some embodiments, the collar 9212 may define or otherwise provide an annular lip 9216 on its bottom surface. The annular lip 9216 may be sized and otherwise configured to mate with or be received into the channel 9206 defined on the inner surface of the mount 9108. In some embodiments, a groove 9218 may be defined on the annular lip 9216 and may be configured to accommodate or otherwise receive a portion of the sensor 9112 extending laterally within the mount 9108. In some embodiments, the collar 9212 may further define or otherwise provide a collar channel 9220 (FIG. 14A) on its upper surface sized to receive and otherwise mate with an annular ridge 9222 (FIG. 14B) defined on the inner surface of the shell 9106 when the sensor control device 9102 is properly assembled.

The sensor 9112 may include a tail 9224 that extends through the second aperture 9202b defined in the mount 9108 to be transcutaneously received beneath a user's skin. The tail 9224 may have an enzyme or other chemistry included thereon to help facilitate analyte monitoring. The sharp 9114 may include a sharp tip 9226 extendable through the first aperture 9202a defined by the shell 9106. As the sharp tip 9226 penetrates the electronics housing 9104, the tail 9224 of the sensor 9112 may be received within a hollow or recessed portion of the sharp tip 9226. The sharp tip 9226 may be configured to penetrate the skin while carrying the tail 9224 to put the active chemistry of the tail 9224 into contact with bodily fluids.

The sensor control device 9102 may provide a sealed subassembly that includes, among other component parts, portions of the shell 9106, the sensor 9112, the sharp 9114, the seal 9208, the collar 9212, and the sensor cap 9120. The sealed subassembly may help isolate the sensor 9112 and the sharp 9114 within the inner chamber 9124 (FIG. 14A) of the sensor cap 9120. In assembling the sealed subassembly, the sharp tip 9226 is advanced through the electronics housing 9104 until the sharp hub 9116 engages the seal 9208 and, more particularly, the first seal element 9210a. The mating member 9118 provided at the bottom of the sharp hub 9116 may extend out the second aperture 9202b in the bottom of the mount 9108, and the sensor cap 9120 may be coupled to the sharp hub 9116 at the mating member 9118. Coupling the sensor cap 9120 to the sharp hub 9116 at the mating member 9118 may urge the first end 9122a of the sensor cap 9120 into sealed engagement with the seal 9208 and, more particularly, into sealed engagement with the second seal element 9210b on the bottom of the mount 9108. In some embodiments, as the sensor cap 9120 is coupled to the sharp hub 9116, a portion of the first end 9122a of the sensor cap 9120 may bottom out (engage) against the bottom of the mount 9108, and the sealed engagement between the sensor hub 9116 and the first seal element 9210a may be able to assume any tolerance variation between features.

FIG. 15 is a cross-sectional side view of the sensor control device 9102, according to one or more embodiments. As indicated above, the sensor control device 9102 may include or otherwise incorporate a sealed subassembly 9302, which may be useful in isolating the sensor 9112 and the sharp 9114 within the inner chamber 9124 of the sensor cap 9120. To assemble the sealed subassembly 9302, the sensor 9112 may be located within the mount 9108 such that the tail 9224 extends through the second aperture 9202b at the bottom of the mount 9108. In at least one embodiment, a locating feature 9304 may be defined on the inner surface of the mount 9108, and the sensor 9112 may define a groove 9306 that is matable with the locating feature 9304 to properly locate the sensor 9112 within the mount 9108.

Once the sensor 9112 is properly located, the collar 9212 may be installed on the mount 9108. More specifically, the collar 9212 may be positioned such that the first seal element 9210a of the seal 9208 is received within the central aperture 9214 defined by the collar 9212 and the first seal element 9210a generates a radial seal against the collar 9212 at the central aperture 9214. Moreover, the annular lip 9216 defined on the collar 9212 may be received within the channel 9206 defined on the mount 9108, and the groove 9218 defined through the annular lip 9216 may be aligned to receive the portion of the sensor 9112 that traverses the channel 9206 laterally within the mount 9108. In some embodiments, an adhesive may be injected into the channel 9206 to secure the collar 9212 to the mount 9108. The adhesive may also facilitate a sealed interface between the two components and generate a seal around the sensor 9112 at the groove 9218, which may isolate the tail 9224 from the interior of the electronics housing 9104.

The shell 9106 may then be mated with or otherwise coupled to the mount 9108. In some embodiments, as illustrated, the shell 9106 may mate with the mount 9108 via a tongue-and-groove engagement 9308 at the outer periphery of the electronics housing 9104. An adhesive may be injected (applied) into the groove portion of the engagement 9308 to secure the shell 9106 to the mount 9108, and also to create a sealed engagement interface. Mating the shell 9106 to the mount 9108 may also cause the annular ridge 9222 defined on the inner surface of the shell 9106 to be received within the collar channel 9220 defined on the upper surface of the collar 9212. In some embodiments, an adhesive may be injected into the collar channel 9220 to secure the shell 9106 to the collar 9212, and also to facilitate a sealed interface between the two components at that location. When the shell 9106 mates with the mount 9108, the first seal element 9210a may extend at least partially through (into) the first aperture 9202a defined in the shell 9106.

The sharp 9114 may then be coupled to the sensor control device 9102 by extending the sharp tip 9226 through the aligned first and second apertures 9202a, b defined in the shell 9106 and the mount 9108, respectively. The sharp 9114 may be advanced until the sharp hub 9116 engages the seal 9208 and, more particularly, engages the first seal element 9210a. The mating member 9118 may extend (protrude) out the second aperture 9202b at the bottom of the mount 9108 when the sharp hub 9116 engages the first seal element 9210a.

The sensor cap 9120 may then be removably coupled to the sensor control device 9102 by threadably mating the internal threads 9128b of the sensor cap 9120 with the external threads 9128a of the mating member 9118. The inner chamber 9124 may be sized and otherwise configured to receive the tail 9224 and the sharp tip 9226 extending from the bottom of the mount 9108. Moreover, the inner chamber 9124 may be sealed to isolate the tail 9224 and the sharp tip 9226 from substances that might adversely interact with the chemistry of the tail 9224. In some embodiments, a desiccant (not shown) may be present within the inner chamber 9124 to maintain proper humidity levels.

Tightening (rotating) the mated engagement between the sensor cap 9120 and the mating member 9118 may urge the first end 9122a of the sensor cap 9120 into sealed engagement with the second seal element 9210b in an axial direction (e.g., along the centerline of the apertures 9202a, b), and may further enhance the sealed interface between the sharp hub 9116 and the first seal element 9210a in the axial direction. Moreover, tightening the mated engagement between the sensor cap 9120 and the mating member 9118 may compress the first seal element 9210a, which may result in an enhanced radial sealed engagement between the first seal element 9210a and the collar 9212 at the central aperture 9214. Accordingly, in at least one embodiment, the first seal element 9210a may help facilitate axial and radial sealed engagements.

As mentioned above, the first and second seal elements 9210a,b may be overmolded onto the mount 9108 and may be physically linked or otherwise interconnected. Consequently, a single injection molding shot may flow through the second aperture 9202b of the mount 9108 to create both ends of the seal 9208. This may prove advantageous in being able to generate multiple sealed interfaces with only a single injection molded shot. An additional advantage of a two-shot molded design, as opposed to using separate elastomeric components (e.g., O-rings, gaskets, etc.), is that the interface between the first and second shots is a reliable bond rather than a mechanical seal. Hence, the effective number of mechanical sealing barriers is effectively cut in half. Moreover, a two-shot component with a single elastomeric shot also has implications to minimizing the number of two-shot components needed to achieve all the necessary sterile barriers. Once properly assembled, the sealed subassembly 9302 may be subjected to a radiation sterilization process to sterilize the sensor 9112 and the sharp 9114. The sealed subassembly 9302 may be subjected to the radiation sterilization prior to or after coupling the sensor cap 9120 to the sharp hub 9116. When sterilized after coupling the sensor cap 9120 to the sharp hub 9116, the sensor cap 9120 may be made of a material that permits the propagation of radiation therethrough. In some embodiments, the sensor cap 9120 may be transparent or translucent, but can otherwise be opaque, without departing from the scope of the disclosure.

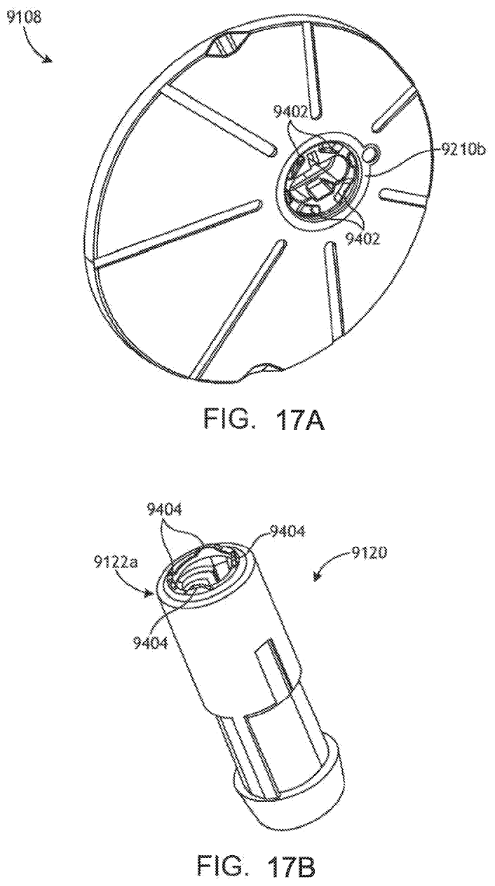

FIG. 16 is an exploded isometric view of a portion of another embodiment of the sensor control device 9102 of FIGS. 13A-13B and 14A-14B. Embodiments included above describe the mount 9108 and the seal 9208 being manufactured via a two-shot injection molding process. In other embodiments, however, as briefly mentioned above, one or both of the seal elements 9210a,b of the seal 9208 may comprise an elastomeric component part independent of the mount 9208. In the illustrated embodiment, for example, the first seal element 9210a may be overmolded onto the collar 9212 and the second seal element 9210b may be overmolded onto the sensor cap 9120. Alternatively, the first and second seal elements 9210a,b may comprise a separate component part, such as a gasket or O-ring positioned on the collar 9212 and the sensor cap 9120, respectively. Tightening (rotating) the mated engagement between the sensor cap 9120 and the mating member 9118 may urge the second seal element 9210b into sealed engagement with the bottom of the mount 9108 in an axial direction, and may enhance a sealed interface between the sharp hub 9116 and the first seal element 9210a in the axial direction. FIG. 17A is an isometric bottom view of the mount 9108, and FIG. 17B is an isometric top view of the sensor cap 9120, according to one or more embodiments. As shown in FIG. 17A, the mount 9108 may provide or otherwise define one or more indentations or pockets 9402 at or near the opening to the second aperture 9202b. As shown in FIG. 17B, the sensor cap 9120 may provide or otherwise define one or more projections 9404 at or near the first end 9122a of the sensor cap 9120. The projections 9404 may be received within the pockets 9402 when the sensor cap 9120 is coupled to the sharp hub 9116 (FIGS. 14A-14B and 93). More specifically, as described above, as the sensor cap 9120 is coupled to the mating member 9118 (FIGS. 14A-14B and 93) of the sensor hub 9116, the first end 9122a of the sensor cap 9120 is brought into sealed engagement with the second seal element 9210b. In this process, the projections 9404 may also be received within the pockets 9402, which may help prevent premature unthreading of the sensor cap 9120 from the sharp hub 9116.

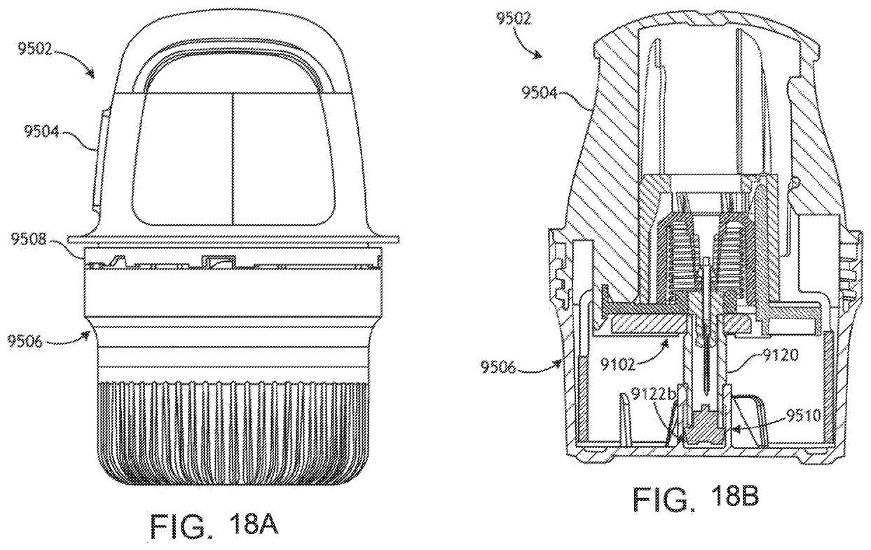

FIGS. 18A and 18B are side and cross-sectional side views, respectively, of an example sensor applicator 9502, according to one or more embodiments. The sensor applicator 9502 may be similar in some respects to the sensor applicator 102 of FIG. 1 and, therefore, may be designed to deliver (fire) a sensor control device, such as the sensor control device 9102. FIG. 18A depicts how the sensor applicator 9502 might be shipped to and received by a user, and FIG. 18B depicts the sensor control device 9102 arranged within the interior of the sensor applicator 9502.

As shown in FIG. 18A, the sensor applicator 9502 includes a housing 9504 and an applicator cap 9506 removably coupled to the housing 9504. In some embodiments, the applicator cap 9506 may be threaded to the housing 9504 and include a tamper ring 9508. Upon rotating (e.g., unscrewing) the applicator cap 9506 relative to the housing 9504, the tamper ring 9508 may shear and thereby free the applicator cap 9506 from the sensor applicator 9502.

In FIG. 18B, the sensor control device 9102 is positioned within the sensor applicator 9502. Once the sensor control device 9102 is fully assembled, it may then be loaded into the sensor applicator 9502 and the applicator cap 9506 may be coupled to the sensor applicator 9502. In some embodiments, the applicator cap 9506 and the housing 9504 may have opposing, matable sets of threads that enable the applicator cap 9506 to be screwed onto the housing 9504 in a clockwise (or counter-clockwise) direction and thereby secure the applicator cap 9506 to the sensor applicator 9502.

Securing the applicator cap 9506 to the housing 9504 may also cause the second end 9122b of the sensor cap 9120 to be received within a cap post 9510 located within the interior of the applicator cap 9506 and extending proximally from the bottom thereof. The cap post 9510 may be configured to receive at least a portion of the sensor cap 9120 as the applicator cap 9506 is coupled to the housing 9504.

Additional details of suitable devices, systems, methods, components and the operation thereof along with related features are set forth in International Publication No. WO2018/136898 to Rao et. al., International Publication No. WO2019/236850 to Thomas et. al., International Publication No. WO2019/236859 to Thomas et. al., International Publication No. WO2019/236876 to Thomas et. al., and U.S. Patent Publication No. 2020/0196919, filed Jun. 6, 2019, each of which is incorporated by reference in its entirety herein. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes. Further details regarding embodiments of sharp modules, sharps, their components, and variants thereof, are described in U.S. Patent Publication No. 2014/0171771, which is incorporated by reference herein in its entirety and for all purposes.

### Exemplary Methods for Detection of Radiological Procedures

Increasingly greater number of patients are incorporating use of continuous glucose monitors into their diabetes management regimens. While continuous glucose monitors (CGM) have been demonstrated to be reliable and accurate, some concerns remain regarding stability of CGMs during radiologic procedures. Indeed, patients who wear CGMs are advised to avoid exposure to whole-body millimeter wave scanners at airport security checkpoints, to avoid putting sensors through baggage x-ray machines and to remove their sensor prior to MRI, CT or X-ray diagnostic procedures. As used here, “CGM” refers to sensor control device 102, and “radiologic procedure” can include exposure to whole-body millimeter wave scanners at airport security checkpoints, exposure to baggage x-ray machines, or user exposure to MRI, CT or X-ray diagnostic procedures.

In an exemplary study, a comprehensive evaluation of the effects of common radiologic procedures of computed tomography (CT), X-ray scanning and MRI on the sensor and sensor control devices was conducted. In particular, evaluation was performed on CGMs having sensor control devices approximately 5 mm thick and 21 mm in diameter (exemplary embodiment A1 and A2) and approximately 3 mm thick and 35 mm in diameter (exemplary embodiment B) and including an analyte sensor for insertion in bodily fluid of a subject.

For CT and X-ray testing, CGM devices were tested under three different radiation exposure conditions: direct radiation, indirect radiation, and scattered radiation. More specifically, for CT, system maximum settings of 140 kV and 360 mAs were used for a total of ten imaging sessions with data collected every 15 minutes. Analyte sensors were evaluated after each exposure for the first three sessions, and then every 15 minutes of sequential exposure until 10 sessions were completed. Similarly, for X-ray, system maximum settings of 150 kV and 500 mAs were used for the first eleven X-ray exposures after which the settings were dropped by 20% to 120 kV and 400 mAs for the remaining nine exposures to address overheating of the X-ray system. Analyte sensors were evaluated after each exposure session (front and side orientations) for the first 3 sessions, and then every 15 minutes of sequential exposures until 10 sessions (20 exposures) were completed.

Acceptance criterion was glucose error of ≤10 mg/dL for nominal glucose readings of 100 mg/dL. All devices passed the functionality testing acceptance criteria in all exposure conditions and orientations after each repeated exposure session at the maximum exposure scan settings allowable by each scanner. Because a single sensor is only on the patient's arm for approximately 14 days and since the maximum exposure conditions were used for each scan, the amount of X-ray and CT radiation exposure is well beyond what a single device would be exposed to clinically. Accordingly, the exemplary study demonstrated CGM sensor functionality is not impacted by X-ray or CT exposure. Furthermore, sensor components were clearly visible with both X-ray and CT imaging modalities, and therefore would allow clinical accounting when reading images. Additionally, there was minimal image artifact created by the CGM.

### Magnetic Resonance Imagining

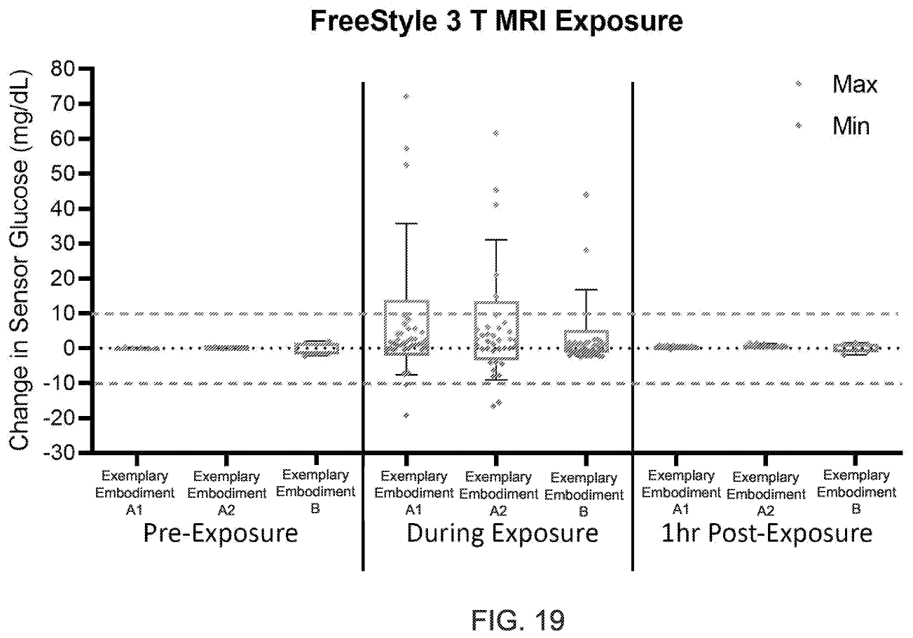

Similarly, CGM functionality testing was performed under an MRI system with a static magnetic field of 1.5 Tesla (1.5 T) and 3 Tesla (3 T) by comparing sensor current before and immediately after MRI exposure. Sensors were also tested for functionality 1 hour and 6 hours after MRI exposure. Acceptance criterion was glucose error of ≤10 mg/dL for nominal glucose readings of 100 mg/dL. Several sensors reported glucose readings outside the acceptance criteria during MRI exposure, however, glucose readings from all sensors returned within the acceptance criteria 1 hour after exposure and remained within their acceptance range 6 hours following MRI exposure. In reference to FIG. 19, the minimum and maximum change in glucose readings from three replicate devices of each device type at the three stages of exposure (pre-, during, and 1 hr post) is shown. Additionally, six different MRI exposure conditions in a 3 T scanner were pooled together to comprise the “during exposure” plots. Within each group, max and min data points are overlaid on the average and standard deviation bars. Similar findings were observed when the devices underwent MRI exposure in a 1.5 T scanner.

Additionally, displacement force testing was conducted near the bore entrance and on the axis of the bore of a 3 T scanner where the spatial gradient of the static magnetic field is known to be maximum. Based on the results of the displacement force testing, the maximum allowable spatial gradients at magnetic field strengths of 1.5 T and 3 T were determined. The displacement forces for exemplary embodiment A1 was 0.132 N, for exemplary embodiment A2 was 0.109 N, which are more than 100× smaller than the force of 15.97 N required to dislodge the sensor control device 102 from the skin of a wearer. Similarly, the displacement forces for exemplary embodiment B was 0.063 N, which, is more than 100× smaller than the force of 7.95 N required to dislodge the sensor control device from the skin of a wearer. These pull-off forces were used to calculate the spatial gradient required to dislodge the sensor and compared to the expected maximum spatial gradient of 19 T/m. The calculated spatial gradients required to dislodge the sensor were found to be 334.9 T/m and 328.2 T/m for exemplary embodiments A and B, respectively, far exceeding what is expected during clinical MRI imaging procedures.

Magnetically induced torque testing was conducted near the isocenter of the 3 T scanner in order to assess the interaction of the maximum uniform static magnetic field with the magnetization in the test sample. Testing was performed in three orientations, based on the qualitative torque results. The acceptance criterion for was defined such that if the test sample did not exhibit discernible torque effects, it could be concluded that the device poses no risk in the MRI environment in terms of device pull-off due to torque. The maximum acceptable magnetically-induced torque of the sensors was set to be less than calculated pull-off torque of the devices, which was calculated by assuming rotation about its central plane and by simplifying the adhesive force to a single point force at the center of mass of the semicircle of the sensor. The resulting pull-off torque was determined to be 0.05 Nm for exemplary embodiment A1 and A2, and 0.018 Nm for exemplary embodiment B.

RF-induced heating evaluations were conducted with temperature probes placed above and below the sensor and at the sensor tip. Calorimetry testing was performed with the phantom filled with saline in the 64 MHz and 128 MHz RF coils, with the sensors aligned with the scanner axis, and the measured and scanner-reported whole-body specific absorption rate (SAR) values were documented for each scan. The acceptance criterion was defined such that the RF-induced temperature rise is less than 6° C. over 15 minutes of scanning in Normal Operating Mode at a whole-body console specific absorption rate (SAR) of 2.0 W/kg, as determined by the Whole Body Modeling Described below. A custom pulsed waveform with a 10 Hz frequency was used for testing. The RF signal was delivered at a frequency of 63.66 MHz for 1.5 T testing and 128.23 MHz for 3 T testing. The input power was set so that the whole body SAR was 2.0 W/kg. The temperature was monitored during the 15 minutes of applied RF signal, as well as 2 minutes before and after the signal. The temperature rise was defined as the difference between the maximum measured temperature and initial temperature. The high electric fields resulted in temperature rises of 2.1° C. at 1.5 T and 2.5° C. at 3 T at a SAR of 2.0 W/kg, well below the acceptance criteria of <6.0° C. As can be seen in FIG. 20, additional evaluation of including 9% and 4.3% experimental uncertainty factors for 1.5 T and 3 T, respectively, the temperature rises were determined to be 2.2° C. at 1.5 T and 2.6° C. at 3 T.

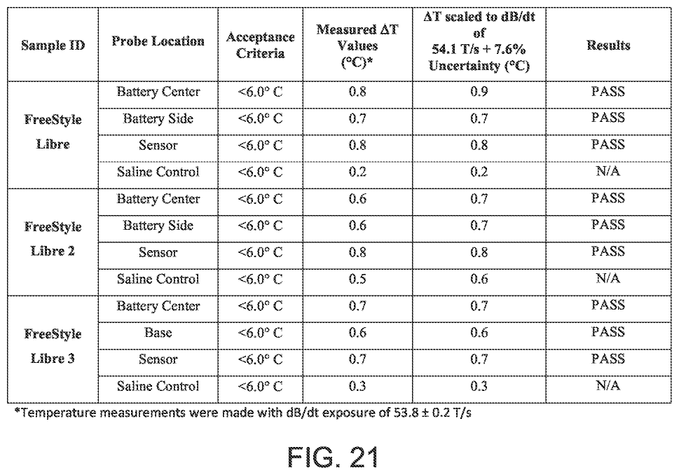

Gradient-induced heating testing was conducted by exposing the sensor control device to a gradient field |dB/dt| rms of 54.1 T/s based on the maximum Normal Operating Mode |dB/dt| RMS exposure for a device at a radial position of 25 cm from isocenter in a scanner with a slew rate of 200 T/m/s, which is approximately the distance of a sensor control device positioned on the lateral upper arm of a wearer. Acceptance criterion for this evaluation was a temperature change <6.0° C. with a gradient coil dB/dt=54 T/s. A sinusoidal waveform with a frequency of 1750 Hz and an amplitude of 1.25 V was used for testing. Temperature probes were placed at locations of highest expected heating based on an initial hotspot evaluation. A fourth probe was used to monitor the bulk temperature in the homogeneous region of the gradient coil at least 2 cm from the device. The temperature was monitored during 15 minutes of applied gradient signal, as well as 2 minutes before and after the signal. The temperature rise was defined as the difference between the maximum measured temperature and initial temperature. The measured temperature change was scaled to a dB/dt of 54.1 T/s. As can be seen in FIG. 21, gradient exposure 54.1 T/s over 15 minutes resulted in a maximum temperature rise of 0.8° C. Including a 7.6% experimental uncertainty factor, the temperature rise was 0.9° C. Human body modeling was performed to confirm predicted safety by scaling the experimental change in temperature (ΔT) values to predicted clinically relevant AT values. An ANSYS® male human body model was used to characterize whole-body and local SAR values. The human body model includes skin, fat, muscles, bones, and organs. Because the glucose sensors are intended for use on the upper arm of a wearer, maximum local SAR values were quantified for a region encompassing the skin up to a 1 cm depth on the side of the upper arm. For local quantitative analysis, a region of interest was defined to encompass the skin of the anterior, lateral, and posterior upper arm with a subdermal depth of up to 1 cm, according to exemplary indications for use. The local SAR values were determined by volume-averaging 10 g of tissue to obtain accurate results, remove numerical artifact, and maintain reasonable computing times. The local SAR values were used for calculating predicted ΔT values. The predicted ΔT values obtained from human body modeling were then scaled to a whole-body SAR of 2.0 W/kg (representing Normal Operating Mode in clinical scanners). These scaled values represent maximum expected heating over at least 15 minutes of scanning with the 2.0 W/kg SAR limitation. The acceptance criterion was defined such that the RF-induced temperature is less than 6° C. over 15 minutes of scanning in Normal Operating Mode at a whole-body console specific absorption rate (SAR) of 2.0 W/kg. As expected, the highest electric fields and levels of SAR occurred in the arms, shoulders, and on the sides of the body when the scanner landmark location was in the proximity of the upper arm. The highest change in temperature was found when the arm was located closest to the magnet bore iscocenter. At 1.5 T, all expected change in temperature values were less than 4 deg c. At 3 T, a maximum scan time limit maintained all delta T values below 4 deg C., including a calculated min cool down time between scans of 6 minutes. It was determined that the highest expected heating after a series of scans and cool down times was a total of 6 deg C. relative to body temperature. When evaluating the region of interest isolation at the same landmark locations, the highest levels of volume-average SAR occurred in the center of the arm at both 1.5 T and 3 T.

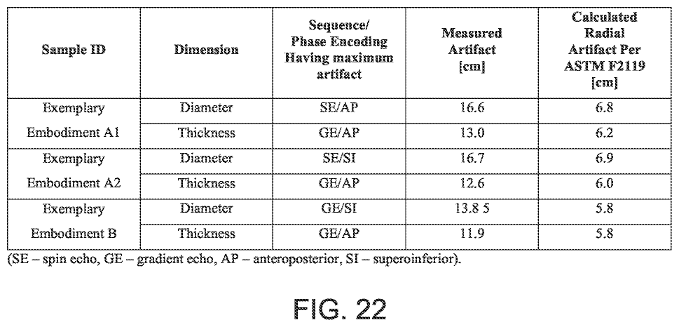

During artifact testing, and as can be seen in FIG. 22, the maximum radial artifact at 3 T standard was determined to be 6.8 cm for the exemplary embodiment A1, 6.9 cm for exemplary embodiment A2, and 5.8 cm for exemplary embodiment B.

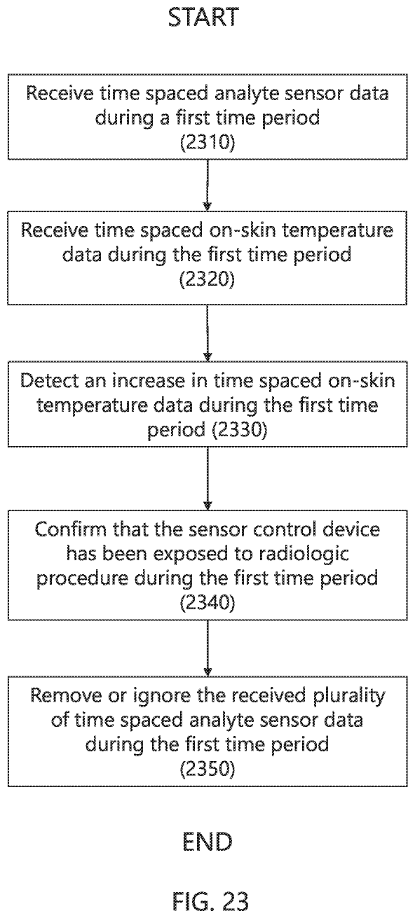

Accordingly, FIG. 23 is a flowchart illustrating a routine associated with determining exposure to radiologic procedure of a sensor control device according to the present disclosure. As shown, in one embodiment, a plurality of time spaced analyte sensor data during a first time period is received (2310). Thereafter, a plurality of time spaced temperature data during the first time period is received (2320). The temperature data can include on-skin temperature, ambient temperature, subcutaneous temperature, sensor-tip temperature, internal body temperature, internal temperature of the sensor control device, etc. Upon detection of an increase in the plurality of time spaced temperature data during the first time period (2330), confirm that the sensor control device has been exposed to radiologic procedure (for example, not limitation, magnetic resonance imagining) during the first time period (2340). In one aspect, increase in the plurality of time spaced temperature data may include a rate of change of the temperature data at or greater than 4 deg C/15 minutes. Within the scope of the present disclosure, the increase in the plurality of time spaced temperature data may include other variations of the rate of change that is greater or less than 4 deg C/15 minutes. Furthermore, while on skin temperature level monitoring and detection is described above, in accordance with aspects of the present disclosure, any suitable body temperature may be measured and used. For example, temperature data can include on-skin temperature, ambient temperature, subcutaneous temperature, sensor-tip temperature, internal body temperature, internal temperature of the sensor control device, etc.

According to embodiments, confirming that the sensor control device has been exposed to a radiologic procedure can include the step of generating an alarm or alert to confirm whether the sensor control device has been exposed to a radiologic procedure, such as an MRI. For example, according to embodiments, in response to the alarm, the user may be required to confirm that the sensor control device has been exposed to a radiologic procedure. The alarm can be auditory, visual, haptic, or any other type of alarm. The alarm can be generated on the on-body unit or the user's computer system (i.e., laptop, desktop, tablet, phablet, smartphone, set-top box, video game console, or other computing device), as described above.

According to embodiments, a prompt can also be provided to the user of the estimated time period for which the on-body unit was subject to radiologic procedure. According to embodiments, the user can confirm or adjust the estimated time period for which the on-body unit was subject to radiologic procedure. For example, upon receiving a prompt of the estimated time period, the user can confirm whether the estimated time period is accurate. Alternatively, the user can adjust (e.g., upwards or downwards) the estimated time period of radiologic procedure.

According to embodiments, confirmation that the sensor control device has been exposed to radiologic procedure can include user-based input. For example, not limitation, upon detection of an increase in the plurality of time spaced temperature data during the first time period, user input may be sought (for example, not limitation, via reader device 120) to confirm whether user had been exposed to a radiologic procedure. According to embodiments, user-based input may be retrospectively retrieved from memory 223 of reader device 120. For example, not limitation, based on user input prior to exposure to radiologic procedure, such as, when a user, prior to exposure to radiologic procedure, inputs a future, anticipated time period when the user expects sensor control device to be exposed to radiologic procedure.

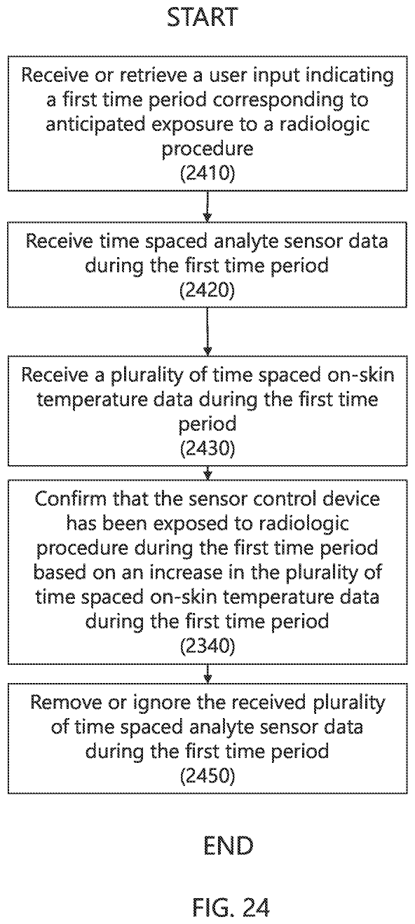

FIG. 24 is a flowchart illustrating a routine associated with determining exposure to radiologic procedure of a sensor control device according to the present disclosure. As shown, in one embodiment, a user input indicating a first time period corresponding to anticipated exposure to a radiologic procedure is retrieved or received (2410). Thereafter, a plurality of time spaced analyte sensor data during the first time period is received (2420). Upon start of the first time period corresponding to anticipated exposure to radiologic procedure, receive a plurality of time spaced temperature data during the first time period is received (2430), wherein the anticipated start of the radiologic procedure corresponds to a start of the first time period. At step 2440, confirm that the sensor control device has been exposed to radiologic procedure (for example, not limitation, magnetic resonance imagining) during the first time period based on an increase in the plurality of time spaced temperature data during the first time period. In one aspect, increase in the plurality of time spaced temperature data which would confirm exposure to a radiologic procedure may include a rate of change of the temperature data at or greater than 4 deg C/15 minutes. Within the scope of the present disclosure, the increase in the plurality of time spaced temperature data may include other variations of the rate of change that is greater or less than 4 deg C/15 minutes. Furthermore, while on skin temperature level monitoring and detection is described above, in accordance with aspects of the present disclosure, any suitable body temperature may be measured and used. For example, temperature data can include on-skin temperature, ambient temperature, subcutaneous temperature, sensor-tip temperature, internal body temperature, internal temperature of the sensor control device, etc.

Referring back to FIGS. 23 and 24, upon confirmation that sensor control device has been exposed to radiologic procedure during the first time period, the received plurality of time spaced analyte sensor data during the first time period may be adjusted. For example, and without limitation, as can be seen in FIGS. 23 and 24, the adjustment can include removing or ignoring the received plurality of time spaced analyte sensor data during the first time period (2350 or 2450). Furthermore, adjusting the received plurality of time spaced analyte sensor data can include adjusting upwards or downwards the received plurality of time spaced analyte sensor data during the first time period. According to embodiments, adjustment can be based on a percentage, an amount, or based upon a function (for example without limitation, linear, cubic, exponential, etc.) of time or function (for example without limitation, linear, cubic, exponential, etc.) of exposure to radiologic procedure. Additionally or alternatively, a notification (visual, auditory, vibratory, or any combination thereof) can be sent to the user via remote device 120 informing the user of potential inaccuracy of the plurality of time spaced analyte sensor date received during the first time period. According to embodiments, analyte sensor data may be removed or ignored up to a predetermined period of time after exposure to radiologic procedure. In one aspect, the predetermined period of time after exposure to radiologic procedure can include one hour, two hours, three hours, etc.

According to embodiments, the routine associated with determining exposure to radiologic procedure of a sensor control device can be performed by processor 166 of sensor control device 102, processing core 206 of reader device 120, or by a network device on network 190.

While monitoring glucose level in addition to monitoring and determining temperature as described in conjunction with the various aspects of the present disclosure, other parameters may be monitored and used to initiate the routine to determine exposure to radiologic procedure. For example, an increase in analyte levels as measured by analyte sensor data during the first time period may be used to determined exposure to radiologic procedure. For example, gradient sensors and/or accelerometers may be used to determine exposure to radiologic procedure. Additional description of gradient sensors and/or accelerometers for determining exposure to radiologic procedures is provided in U.S. Patent Publication No. 2020/0188678A1, filed Feb. 24, 2020, and U.S. Pat. No. 10,668,292, Jun. 2, 2020, disclosures of each of which are incorporated herein by reference.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1. A method, comprising:
receiving a plurality of analyte data over a first time period monitored by an analyte sensor in fluid contact with bodily fluid under a skin surface, the plurality of analyte data corresponding to an analyte level;
receiving a plurality of temperature data over the first time period from a temperature sensor;
determining a rate of change of the plurality of temperature data over the first time period;
if the determined rate of change of the plurality of temperature data is above a predetermined threshold, receiving user input to confirm exposure to a radiologic procedure during the first time period; and
adjusting the plurality of analyte data over the first time period based on the confirmed exposure to radiologic procedure.

2. The method of claim 1, wherein the time period includes one hour.

3. The method of claim 1, wherein the predetermined threshold is 4 degrees Celsius over 15 minutes.

4. The method of claim 1, wherein the user input is received via a reader device.

5. The method of claim 1, wherein adjusting the plurality of analyte data includes removing the plurality of analyte data for the first time period.

6. The method of claim 1, wherein adjusting the plurality of analyte data includes ignoring the plurality of analyte data for the first time period.

7. The method of claim 1, wherein the temperature data comprises an on-skin temperature data.

8. The method of claim 1, wherein confirming exposure to the radiologic procedure comprises generating an alarm.

9. A method, comprising:
receiving a user input indicating a first time period corresponding to anticipated exposure to a radiologic procedure;
receiving a plurality of analyte data over the first time period monitored by an analyte sensor in fluid contact with bodily fluid under a skin surface, the plurality of analyte data corresponding to an analyte level;
receiving a plurality of temperature data over the first time period for the skin surface from a temperature sensor;
determining a rate of change of the plurality of temperature data over the first time period;
confirming exposure to radiologic procedure during the first time period if the determined rate of change of the plurality of temperature data is above a predetermined threshold; and
adjusting the plurality of analyte data over the first time period based on the confirmed exposure to radiologic procedure.

10. The method of claim 9, wherein the first time period includes one hour.

11. The method of claim 9, wherein the predetermined threshold is 4 degrees Celsius over 15 minutes.

12. The method of claim 9, wherein the user input is received via a reader device.

13. The method of claim 9, wherein adjusting the plurality of analyte data includes removing the plurality of analyte data for the first time period.

14. The method of claim 9, wherein adjusting the plurality of analyte data includes ignoring the plurality of analyte data for the first time period.

15. The method of claim 9, wherein the temperature data comprises an on-skin temperature data.

16. The method of claim 9, wherein confirming exposure to the radiologic procedure comprises generating an alarm.

17. The method of claim 9, wherein the temperature data received from the temperature sensor is at least of on-skin temperature, ambient temperature, subcutaneous temperature, sensor-tip temperature, internal body temperature, of internal temperature of a sensor control device of the analyte sensor.

18. The method of claim 8, wherein the alarm is at least one of an auditory, visual, or haptic alarm.

19. The method of claim 1, wherein the temperature data received from the temperature sensor is at least of on-skin temperature, ambient temperature, subcutaneous temperature, sensor-tip temperature, internal body temperature.

20. An analyte monitoring system comprising:
an analyte sensor configured to generate data indicative of an analyte level;
a temperature sensor configured to generate temperature data; and
one or more processors operably coupled to the analyte sensor and the temperature sensor, the one or more processors operably coupled to a memory storing instructions, that when executed, cause the processor to perform a method including:
receiving a plurality of analyte data over a first time period from the analyte sensor, the plurality of analyte data corresponding to an analyte level;
receiving a plurality of temperature data over the first time period from the temperature sensor;
determining a rate of change of the plurality of temperature data over the first time period;
if the determined rate of change of the plurality of temperature data is above a predetermined threshold, receiving user input to confirm exposure to a radiologic procedure during the first time period; and
adjusting the plurality of analyte data over the first time period based on the confirmed exposure to radiologic procedure.
